SEQUENCE LISTING (28 sequences)

SEQ ID NO: 1 - DNA, 20 bp, Artificial
  oligonucleotide labelling agent
ttctcactactattagtact

SEQ ID NO: 2 - DNA, 17 bp, Artificial
  oligonucleotide labelling agent
agtactaatagtagtga

SEQ ID NO: 3 - DNA, 18 bp, Artificial
  oligonucleotide labelling agent
gattggtccactaacgta

SEQ ID NO: 4 - DNA, 15 bp, Artificial
  oligonucleotide labelling agent
tacgttagtggacca

SEQ ID NO: 5 - DNA, 17 bp, Artificial
  oligonucleotide labelling agent
tccaaccaaactgaccg

SEQ ID NO: 6 - DNA, 14 bp, Artificial
  oligonucleotide labelling agent
cggtcagtttggtt

SEQ ID NO: 7 - DNA, 17 bp, Artificial
  oligonucleotide labelling agent
tcgcaaggaactaccga

SEQ ID NO: 8 - DNA, 14 bp, Artificial
  oligonucleotide labelling agent
tcggtagttccttg

SEQ ID NO: 9 - DNA, 18 bp, Artificial
  oligonucleotide labelling agent
aggtggatgtgttacgat

SEQ ID NO: 10 - DNA, 15 bp, Artificial
  oligonucleotide labelling agent
atcgtaacacatcca

SEQ ID NO: 11 - DNA, 17 bp, Artificial
  oligonucleotide labelling agent
cgcggtttcctcagaca

SEQ ID NO: 12 - DNA, 14 bp, Artificial
  oligonucleotide labelling agent
tgtctgaggaaacc

SEQ ID NO: 13 - DNA, 18 bp, Artificial
  oligonucleotide labelling agent
gtacacatgagcgaatca

SEQ ID NO: 14 - DNA, 15 bp, Artificial
  oligonucleotide labelling agent
tgattcgctcatgtg

SEQ ID NO: 15 - DNA, 18 bp, Artificial
  oligonucleotide labelling agent
cactctcccattagtcgg

SEQ ID NO: 16 - DNA, 15 bp, Artificial
  oligonucleotide labelling agent
ccgactaatgggaga

SEQ ID NO: 17 - DNA, 15 bp, Artificial
  oligonucleotide labelling agent
gccgacgacgaaggc

SEQ ID NO: 18 - DNA, 12 bp, Artificial
  oligonucleotide labelling agent
gccttcgtcgtc

SEQ ID NO: 19 - DNA, 20 bp, Artificial
  oligonucleotide labelling agent
gcccgctaagatattctaag

SEQ ID NO: 20 - DNA, 17 bp, Artificial
  oligonucleotide labelling agent
cttagaatatcttagcg

SEQ ID NO: 21 - DNA, 15 bp, Artificial
  oligonucleotide labelling agent
gacaacgcgacggat

SEQ ID NO: 22 - DNA, 12 bp, Artificial
  oligonucleotide labelling agent
atccgtcgcgtt

SEQ ID NO: 23 - DNA, 22 bp, Artificial
  oligonucleotide labelling agent
gttgataaatattttacagagt

SEQ ID NO: 24 - DNA, 19 bp, Artificial
  oligonucleotide labelling agent
actctgtaaaatatttatc

SEQ ID NO: 25 - DNA, 19 bp, Artificial
  oligonucleotide labelling agent
gacgcttattatggacttc

SEQ ID NO: 26 - DNA, 16 bp, Artificial
  oligonucleotide labelling agent
gaagtccataataagc

SEQ ID NO: 27 - DNA, 18 bp, Artificial
  oligonucleotide labelling agent
acaaagcggtcttacggt

SEQ ID NO: 28 - DNA, 15 bp, Artificial
  oligonucleotide labelling agent
accgtaagaccgctt

(19) United States
(12) Patent Application Publication
(10) Pub. No.: US 2022/0128565 A1
(43) Pub. Date: Apr. 28, 2022

(54) ANALYTE DETECTION BY SELECTIVE LABELING OF BIOLOGICAL SAMPLES

(71) Applicant: Akoya Biosciences, Inc., Menlo Park, CA (US)

(72) Inventors: Peter J. Miller, Cambridge, MA (US); Julia Kennedy-Darling, Redwood City, CA (US); Yi Zheng, Dover, MA (US); Clifford C. Hoyt, Wellesley, MA (US); Gajalakshmi Dakshinamoorthy, Menlo Park, CA (US)

(21) Appl. No.: 17/428,410

(22) PCT Filed: Feb. 4, 2020
(86) PCT No.: PCT/US2020/016667

§ 371 Date: Aug. 4, 2021

Related U.S. Application Data
(60) Provisional application No. 62/801,011, filed on Feb. 4, 2019.
(60) Provisional application No. 62/801,009, filed on Feb. 4, 2019.

(51) Int. Cl. G01N 33/58; C12Q 1/682

(52) U.S. Cl. CPC G01N 33/582; G01N 2458/10; C12Q 1/682

(57) ABSTRACT

The disclosure features methods that include: contacting a biological sample having a first target analyte with a first agent, where the first agent includes a first binding species that specifically binds to the first target analyte, and a first oligonucleotide conjugated to the binding species; contacting the biological sample with a second agent, where the second agent includes a first reactive species and a second oligonucleotide conjugated to the first reactive species, to hybridize at least a portion of the second oligonucleotide to at least a portion of the first oligonucleotide; and contacting the biological sample with a first labeling species, where the first labeling species reacts with the first reactive species to deposit the first labeling species or a derivative thereof in the biological sample.

### CROSS-REFERENCE TO RELATED APPLICATIONS

This application claims priority to U.S. Provisional Patent Application No. 62/801,011, filed on Feb. 4, 2019, and to U.S. Provisional Patent Application No. 62/801,009, filed on Feb. 4, 2019, the entire contents of each of which are incorporated herein by reference.

### TECHNICAL FIELD

This disclosure relates to the detection of biological analytes in a sample by selective labeling of the sample.

### BACKGROUND

Antibodies have been used to selectively bind to a wide variety of proteins in biological samples. Immunohistochemical methods typical involve binding a dye-conjugated antibody to a complementary marker, thereby labeling the marker with the dye. Detection of fluorescence emission from the dye label reveals the presence of the marker in the sample.

### SUMMARY

This disclosure features methods for selectively applying dyes and other labeling species to samples to identify and quantify specific target analytes in the samples. Following identification and/or quantification, additional dyes and labeling agents can be added to the sample to identify and quantify additional specific target analytes. In such a manner, a series of serial labeling and detection cycles can be performed to selectively identify and quantify specific target analytes in a sample.

The methods can be performed by binding multiple first agents to a sample, where each first agent includes a binding species that specifically binds to different a target analyte in the sample, and a different first oligonucleotide conjugated to the binding species. A second agent is then introduced, and includes a second oligonucleotide conjugated to a reactive species. The second oligonucleotide hybridizes to the first oligonucleotide of one of the first agents, localizing the second agent in the sample at positions corresponding to the target analyte associated with that first agent. A reaction between the reactive species and a labeling agent that is introduced deposits the labeling agent in proximity to that target analyte. The second agent can then be removed by dehybridization under relatively mild conditions, ensuring that each of the first agents remains bound to the sample. Subsequently, additional labeling cycles can be performed in which different second agents are introduced, each of which includes a different second oligonucleotide conjugated to a reactive species. By selecting second agents with second oligonucleotides that are complementary to specific first oligonucleotides, specific target analytes can be selectively labeled with different labeling agents. The relatively mild conditions under which the second agents are removed from the sample ensures that the first agents remain bound to the sample, and sample integrity is maintained.

In one aspect, the disclosure features methods that include: (i) contacting a biological sample comprising a first target analyte with a first agent, wherein the first agent comprises a first binding species that specifically binds to the first target analyte, and a first oligonucleotide conjugated to the binding species; (ii) contacting the biological sample with a second agent, wherein the second agent comprises a first reactive species and a second oligonucleotide conjugated to the first reactive species, to hybridize at least a portion of the second oligonucleotide to at least a portion of the first oligonucleotide; (iii) contacting the biological sample with a first labeling species, wherein the first labeling species reacts with the first reactive species to deposit the first labeling species or a derivative thereof in the biological sample; (iv) removing the second agent from the biological sample following deposition of the first labeling species or the derivative thereof; (v) contacting the biological sample with a third agent, wherein the third agent comprises a second binding species that specifically binds to a second target analyte in the biological sample, and a third oligonucleotide conjugated to the second binding species; (vi) contacting the biological sample with a fourth agent, wherein the fourth agent comprises a second reactive species and a fourth oligonucleotide conjugated to the second reactive species, to hybridize at least a portion of the fourth oligonucleotide to at least a portion of the third oligonucleotide; and (vii) contacting the biological sample with a second labeling species, wherein the second labeling species reacts with the second reactive species to deposit the second labeling species or a derivative thereof in the biological sample.

Embodiments of the methods can include any one or more of the following features.

The first reactive species can include a catalytic agent, e.g., an enzyme, such as horseradish peroxidase. The first labeling species can include a dye. The first labeling species can include a conjugate of an inactive tyramide or a derivative thereof and a dye.

Contacting the biological sample with the first labeling species can include converting the first labeling species to a conjugate of an active tyramide or a derivative thereof and the dye, where the active tyramide or a derivative thereof binds to the biological sample in proximity to the second agent.

The first binding species can include an antibody or an antibody fragment.

The first oligonucleotide and/or the second oligonucleotide can include at least 10 nucleotides. Nucleotide sequences of the first and second oligonucleotides can be at least 70% complementary. The second oligonucleotide can include a larger number of nucleotides than the first oligonucleotide.

The second oligonucleotide can include multiple contiguous, non-consecutive nucleotide sequences that are complementary to different portions of a sequence of the first oligonucleotide.

The first and second reactive species can be the same. The first and second reactive species can each include an enzyme, e.g., horseradish peroxidase.

The first and third oligonucleotides can be different. The second and fourth oligonucleotides can be different.

The first labeling species can include a first dye, and the second labeling species can include a second dye different from the first dye.

The first binding species can include a first antibody or a first antibody fragment, and the second binding species can include a second antibody or a second antibody fragment, and the first and second binding species can selectively bind to different first and second target analytes in the biological sample.

The first oligonucleotide can include a nucleotide sequence of RNA bases, and/or a nucleotide sequence of DNA bases. The first oligonucleotide can include at least one synthetic nucleotide. The first oligonucleotide can be fully single-stranded, or alternatively, partially double-stranded.

The dye can include a chromogenic species or a fluorescent species.

Embodiments of the methods can also include any of the other features described herein, including any combinations of features even if described in connection with different embodiments, unless expressly stated otherwise.

In another aspect, the disclosure features reagent kits that include: a first agent, wherein the first agent comprises a first binding species that specifically binds to a first target analyte of a biological sample, and a first oligonucleotide conjugated to the first binding species; a second agent, wherein the second agent comprises a second binding species that specifically binds to a second target analyte of the biological sample, and a second oligonucleotide conjugated to the second binding species; a third agent, wherein the third agent comprises a reactive species and a third oligonucleotide conjugated to the reactive species; a fourth agent, wherein the fourth agent comprises the reactive species and a fourth oligonucleotide conjugated to the reactive species; a first labeling species; and a second labeling species, where the first and second labeling species each react with the reactive species to deposit the first and second labeling species or a derivative thereof, respectively, in the biological sample.

Embodiments of the reagent kits can include any of the features described herein, including any combinations of features even if described in connection with different embodiments, unless expressly stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1 is a schematic diagram showing a series of example steps for analysis of a biological sample.

FIGS. 2A-2D are schematic diagrams showing steps of a method for sample analysis.

FIG. 3A is a flow chart showing a series of example steps for analysis of a biological sample.

FIGS. 3B-3E are schematic diagrams showing steps of a method for sample analysis.

FIGS. 4A-4E are schematic diagrams showing steps of a method for sample analysis.

FIGS. 5A-5D are schematic diagrams showing examples of oligonucleotides.

FIG. 5E is a schematic diagram showing an example first agent.

FIG. 5F is a schematic diagram showing an example second agent.

FIG. 6 is a schematic diagram showing an example multispectral imaging system.

FIG. 7 is a schematic diagram showing an example controller.

FIGS. SA-8D are images of a tissue section labeled with different labeling moieties.

FIGS. 9A-9C are images of a tissue section labeled with different reporting agents and labeling moieties.

FIGS. 10A-10F are images of a tissue section labeled with different labeling moieties.

Like reference symbols in the various drawings indicate like elements.

### DETAILED DESCRIPTION

### Introduction

Analysis of multiple target analytes in a biological sample is an important aspect of modern research methods. For example, by identifying and quantifying multiple disease markers, complex regulation and co-expression relationships in cells can be elucidated, which allows models of disease progression to be validated, and assists development of targeted therapies to interfere with key aspects of that progression.

A variety of different techniques have been used to identify and quantify multiple target analytes in biological samples. For example, techniques for targeting multiple expressed biomarkers (e.g., antigens, peptides) include exposing the sample to a set of probes, each of which includes an antibody that specifically binds to one of the biomarkers and is conjugated to a different dye species. To target N different biomarkers in a sample, N different types of probes are introduced, each of which stains one type of biomarker with one of N different dyes. Emission (e.g., fluorescence) from each of the different probe dyes is then measured to identify and quantify the N different biomarkers in the sample.

In such methods, emission from each of the different probe dyes is distinguished to separately identify and quantify each of the different biomarkers targeted by the probes. In practical terms, this can impose an effective limit on the extent to which multiple biomarkers can be analyzed, as it can be increasingly difficult to separate and quantify emission measurements corresponding to different probe dyes as the number of such dyes in a sample increases. For example, even with strategic selection of probe dyes and relatively sophisticated methods for decomposing fluorescence emission measurements into contributions from individual component dye emission spectra, the foregoing methods can be limited to simultaneous multiplexed interrogation of about ten different biomarkers in a sample.

The foregoing immunohistochemical methods for target analyte labeling can also be limited by the quantity of probe dye that can be delivered to specifically label each of the target analytes in the sample. Because probe dyes are conjugated to specific antibodies, the amount of probe dye that can be applied to a specific location in a sample depends directly on the number of probe dye moieties that are directly attached to an antibody that binds to an analyte at that location. Certain immunohistochemical methods are therefore somewhat limited in their ability to amplify signals arising from particular target analytes by selectively depositing larger quantities of probe dye at locations corresponding to those target analytes.

To further extend the number of target analytes in a sample that can be identified and quantified, conventional histochemical labeling methods can be modified to include an antibody removal step. For example, in a first around of sample analysis, a set of N different dye-conjugated antibodies can be used to label a sample, and N different biomarkers corresponding to the N different probes can be identified and quantified in the sample by measuring emission from the N different dyes. Then, an antibody removal step can be performed in which the N different probes are removed from the sample. Subsequently, a new set of M different dye-conjugated antibodies is used to label the sample, where the M antibodies label different target analytes than the prior N probes. Measuring emission from the M different dyes of this second set of probes leads to identification and quantification of a second set of M different biomarkers in the sample. This methodology can be extended with multiple cycles of antibody removal and multiplexed labeling.

However, the preparative conditions used to remove bound probe antibodies from the sample can be relatively time-consuming, aggressive, and can adversely affect the integrity of certain types of samples. Accordingly, the number of such labeling and antibody removal cycles that can be carried out before the sample is damaged can be limited. Further, depending upon the nature of binding between specific antibodies and corresponding biomarkers in the sample, it can be difficult to fully remove each of the probes from the sample following each analytical cycle. If a probe is not fully removed from the sample, then a certain amount of its corresponding dye label remains in the sample. Emission from the remaining dye label in subsequent analytical cycles can interfere with emission from dyes conjugated to other probes, leading to inaccurate quantification of certain biomarkers in the sample. Thus, the number of multiplexed labeling and detection cycles that can effectively be performed may be limited by the extent to which dye-conjugated, antibody-based probes can be removed from the sample.

In certain conventional analytical procedures, dye-quenching methods are used to extinguish signals arising from residual antibody-conjugated dyes in a sample. However, these methods too can be difficult and time-consuming to implement. For example, it can be challenging to apply dye quenching reagents that fully extinguish contributions from residual antibody-conjugated dyes, and yet do not interfere with signals generated by subsequently applied antibody-conjugated dyes, and also do not exert undue influence on the biochemical, structural, and spectral properties of the sample.

This disclosure features methods for performing multiplexed labeling, identification, signal amplification, and quantification of target analytes in a biological sample. The methods can be used to perform multiple cycles of target analyte labeling, detection, and removal of certain agents involved in the labeling process, without disrupting antibody-biomarker binding in the sample. Instead, removal of agents involved in the labeling process is performed by dehybridizing the agents under relatively mild conditions, preserving sample integrity and ensuring that removal of the agents during each labeling and detection cycle is nearly complete. As a result, little or no cross-species labeling occurs. Instead, each target analyte in the sample can be selectively labeled with a different labeling species, and deposition of each labeling species is highly constrained to regions of the sample that specifically correspond to the location of a specific target analyte.

### Analysis of Target Analytes

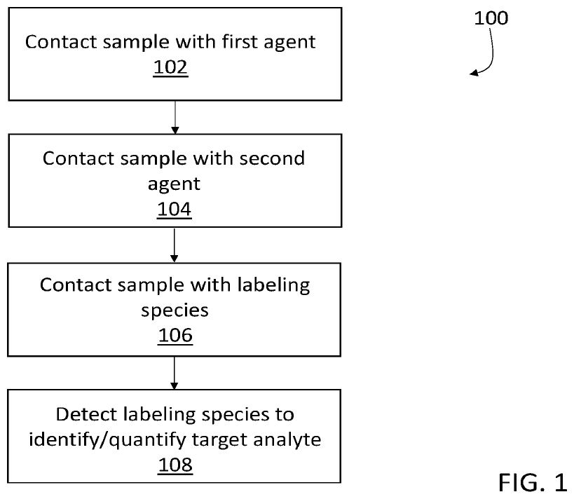

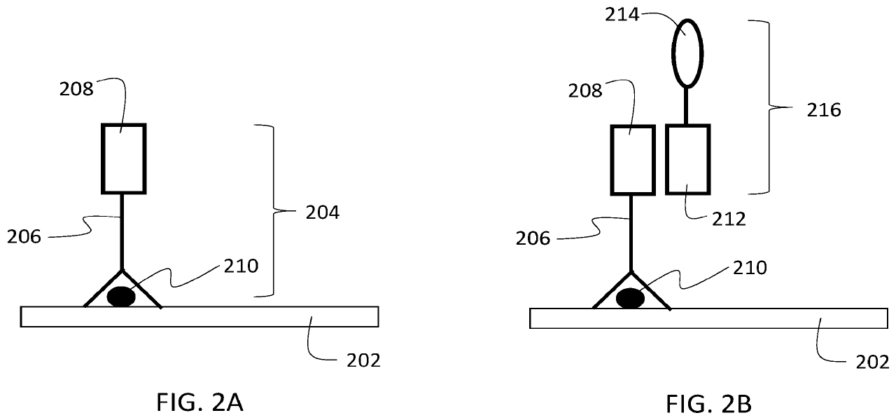

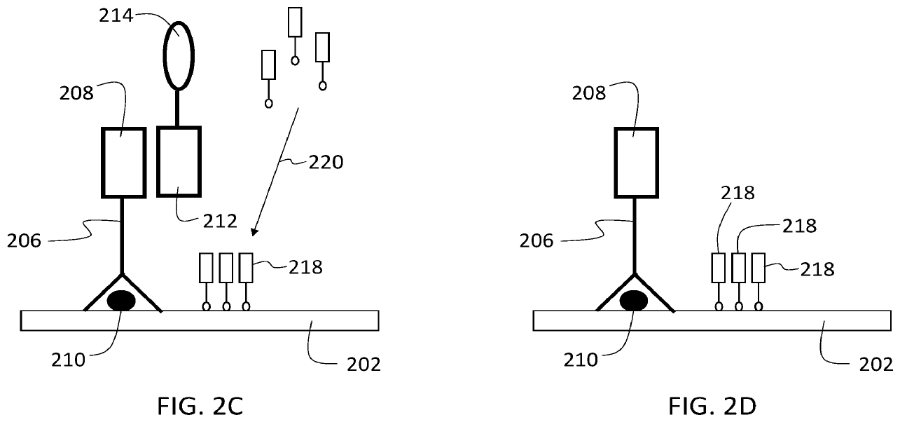

This disclosure describes a variety of different analytical methodologies for identifying and quantifying multiple target analytes in a biological sample. FIG. 1 is a flow chart 100 showing a series of example steps for implementing one method of sample analysis. In a first step 102, a biological sample that includes a target analyte is contacted with a first agent that specifically binds to the target analyte. This first step is illustrated schematically in FIG. 2A. In FIG. 2A, a biological sample 202 includes a target analyte 210. Sample 202 is contacted with a first agent 204. First agent 204 includes a binding species 206 that specifically binds to target analyte 210, and a first oligonucleotide 208 that is conjugated to binding species 206. In this manner, first agent 204 specifically localizes at positions in the sample that correspond to target analyte 210.

As used herein, the terms “contacts” and “contacting” mean that an agent, species, moiety, or other element is brought into association with a sample, or another agent, species, moiety, or element, such that the two interact with one another. For example, when sample 202 is “contacted” with the first and second agents, labeling species, and reporting agents, these agents and species are brought into close enough association with the sample that they interact with the sample, and can bind with the sample or with other agents, species, moieties, and elements that have previously been contacted to, bound to, hybridized to, and/or deposited in, the sample.

Returning to FIG. 1, in a next step 104, the sample is contacted with a second agent that associates with the first agent. This step is illustrated schematically in FIG. 2B. In FIG. 2B, a second agent 216 contacts sample 202. Second agent 216 includes a second oligonucleotide 212 conjugated to a reactive species 214. Second oligonucleotide 212 is at least partially complementary to first oligonucleotide 208, so that the first and second oligonucleotides hybridize. In this manner, second agent 216 localizes in the sample at the same positions as first agent 204, and therefore, at the positions corresponding to target analyte 210.

Returning again to FIG. 1, in step 106, the sample is contacted with a labeling species. The labeling species reacts with the reactive species of the second agent from step 104, depositing the labeling species in the sample at locations in proximity to the second agent. This step is illustrated schematically in FIG. 2C, in which a labeling species 218 contacts the sample. As shown in FIG. 2C, labeling species 218 reacts with reactive species 214 in a reaction represented by arrow 220. The reaction deposits labeling species 218 or a derivative thereof in the sample at locations 222 in proximity to the second agent 216, and therefore, at locations in proximity to target analyte 210. In this manner, the deposited labeling species 218 (or a derivative thereof) is spatially co-localized with target analyte 210.

Again referring to FIG. 1, after deposition of the labeling species or a derivative thereof in step 106, the labeling species is detected in step 108 to identify and/or quantify target analyte 210 in sample 202. Following detection of the labeling species or a derivative thereof, the procedure shown in flow chart 100 ends.

The foregoing procedure and the other methods described herein can be used to identify and quantify a wide variety of different analytes 210 in biological sample 202. Examples of analytes 210 include, but are not limited to, antigens, peptides, proteins, and other amino-acid containing moieties. Additional examples of analytes 210 include, but are not limited to, oligonucleotides, including oligonucleotides containing DNA bases, RNA bases, both DNA and RNA bases, and synthetic bases, nucleic acid fragments, and lipids.

The methods described herein are suited for the identification and quantification of many different clinically relevant biomarkers in biological samples, particularly biomarkers that are expressed in tumor tissues, in the tumor microenvironment, and tissues representative of other disease states. Examples of such biomarkers that correspond to analytes 210 include, but are not limited to, tumor markers such as Sox10, S100, pan-cytokeratin, PAX5, PAX8; immune cell identifiers such as CD3, CD4, CD8, CD20, FoxP3, CD45RA, CD45LCA, CD68, CD163, CD11c, CD33, HLADR; activation markers such as Ki67, granzyme B; checkpoint-related markers such as TIM3, LAG3, PD1, PDL1, CTLA4, CD80, CD86, IDO-1, VISTA, CD47, CD26.

The methods described herein can be used to analyze a variety of different types of biological samples 202. In some embodiments, the biological sample 202 can be fresh, frozen, or fixed. The biological sample can be of animal origin, such as from a human, mouse, rat, cow, pig, sheep, monkey, rabbit, fruit fly, frog, nematode or woodchuck. The biological sample can include formalin-fixed paraffin-embedded (FFPE) tissue sections, frozen tissue sections, fresh tissue, cells obtained from a subject (e.g., via fine-needle aspirate or other technique), cultured cells, biological tissue, biological fluid, a homogenate, or an unknown biological sample.

In certain embodiments, the biological sample 202 can be immobilized on a surface. For example, the surface can be a slide, a plate, a well, a tube, a membrane, or a film. In some embodiments, the biological sample 202 can be mounted on a slide. In certain embodiments, the biological sample 202 can be fixed using a fixative, such as an aldehyde, an alcohol, an oxidizing agent, a mercurial, a picrate, HOPE fixative, or another fixative. The biological sample may alternatively, or in addition, be fixed using heat fixation. Fixation can also be achieved via immersion or perfusion.

In some embodiments, the biological sample 202 can be frozen. For example, the biological sample can be frozen at less than 0° C., less than −10° C., less than −20° C., less than −30° C., less than −40° C., less than −50° C., less than −60° C., less than −70° C., or less than −80° C.

In certain embodiments, the biological sample 202 can be immobilized in a three dimensional form. The three dimensional form can include, for example, a frozen block, a paraffin block, or a frozen liquid. For example, the biological sample 202 can be a block of frozen animal tissue in an optimal cutting temperature compound. The block of tissue can be frozen or fixed. In some embodiments, the block of tissue can be cut to reveal a surface which can be the surface contacted by first agent as discussed above.

In some embodiments, where the biological sample 202 corresponds to a block, the block can be sliced to produce serial sections of the block, each of which can be analyzed according to the methods described herein. By doing so, three dimensional information (e.g., information as a function of depth within the sample) about the identity and/or quantity of one or more target analytes in the sample can be obtained.

In general, the binding species 206 is selected to target a specific analyte in sample 202. The methods described herein can be implemented with a wide variety of different types of binding species. For example, to target specific antigens, peptides, proteins, or other amino acid-containing species in sample 202, binding species 206 can include an antibody or antibody fragment. The antibody or antibody fragment can include any one of different types of antibody species, including but not limited to, an immunoglobulin G (IgG), an immunoglobulin M (IgM), a polyclonal antibody, a monoclonal antibody, a single-chain fragment variable (scFv) antibody, a nanobody, an antigen-binding fragment (Fab), and a diabody. Antibodies and antibody fragments can be of mouse, rat, rabbit, human, camelid, or goat origin. In some embodiments, the antibody or antibody fragment can be raised against a human, mouse, rat, cow, pig, sheep, monkey, rabbit, fruit fly, frog, nematode or woodchuck antigen. In certain embodiments, the antibody or antibody fragment can be raised against an animal, plant, bacteria, fungus, or protist antigen.

A variety of different binding mechanisms can occur between binding species 206 and target analyte 210 in sample 202. In some embodiments, for example, binding species 206 (e.g., an antibody or antibody fragment) binds reversibly to target analyte 210. In certain embodiments, binding species 206 binds irreversibly to target analyte 210. Binding between binding species 206 and target analyte 210 can occur via the formation of one or more covalent bonds in some embodiments. Alternatively, or in addition, binding species 206 and target analyte 210 bind via one or more non-covalent bonds. One or more fixing agents can be used to promote the formation of covalent and or non-covalent bonds.

The specific binding mechanism that occurs between binding species 206 and target analyte 210 in sample 202 depends on the nature of binding species 206 and target analyte 210. For example, where binding species 206 is an antibody or antibody fragment and target analyte 210 is an antigen, binding occurs between the antigen epitope and the paratope of the antibody or antibody fragment. As another example, wherein binding species 206 is an antibody or antibody fragment and target analyte 210 is a lipid, binding can occur between a recognition site on the antibody or antibody fragment and a head group of the lipid (e.g., a phospholipid head group).

In general, binding species 206 binds to target analyte 210 in sample 202 with a particular sensitivity, which refers to the statistical fraction of target analyte 210 entities in a sample that are correctly recognized and bound by binding species 206. In some embodiments, the sensitivity of binding species 206 for target analyte 210 is 60% or more (e.g., 70% or more, 75% or more, 80% or more, 85% or more, 90% or more, 95% or more, 99% or more).

Binding species 206 also generally binds to target analyte 210 with a particular specificity, which refers to the statistical rate or efficiency with which the binding species selectively binds to a particular target analyte 210 in preference to other target analytes in a biological sample. In some embodiments, binding species 206 has a specificity for target analyte 210 of at least 60% (e.g., at least 65%, at least 70%, at least 75%, at least 80%, at least 85%, at least 90%, at least 95%, at least 99%).

The affinity of binding species 206 for target analyte 210 generally refers to the strength of binding between binding species 206 and target analyte 210, and can be characterized by a dissociation constant K_{d}. In some embodiments, the affinity of binding species 206 for target analyte 210 is characterized by a dissociation constant of no more than 10⁻⁴M (e.g., no more than 10⁻⁵ M, no more than 10⁻⁶ M, no more than 10⁻⁷ M, no more than 10⁻⁸ M, no more than 10⁻⁹M, no more than 10⁻¹⁰ M, no more than 10⁻¹¹M, no more than 10⁻¹² M, no more than 10⁻¹³ M, no more than 10⁻¹⁴ M).

In some embodiments, binding species 206 binds to at least 20% (e.g., at least 30%, at least 40%, at least 50%, at least 60%, at least 70%, at least 80%, at least 90%) of target analyte 210 in sample 202. As discussed above, in general, binding species 206 selectively binds to target analyte 210 in sample 202. As used herein, “selective binding” means that at least 70% or more (e.g., 80% or more, 90% or more, 95% or more) of binding species 206 in sample 202 is bound to target analyte 210 rather than to other species in sample 202.

### Oligonucleotides and Hybridization

In general, the first oligonucleotide 208 includes multiple nucleotides. The nucleotides can include, for example, DNA bases (e.g., A, C, G, T), RNA bases (e.g., A, C, G, U), and any combination for DNA and/or RNA bases. First oligonucleotide 208 can also include non-natural (e.g., synthetic) nucleotides, including DNA analogues and/or RNA analogues. Examples of such synthetic analogues include, but are not limited to, peptide nucleic acids, morpholino and locked nucleic acids, glycol nucleic acids, and threose nucleic acids.

The sequence of bases in first oligonucleotide 208 can generally be any sequence. Moreover, in general, nucleotides and other moieties in first oligonucleotide 208 can be conjugated via natural and/or non-natural (e.g., synthetic) linkages.

In some embodiments, first oligonucleotide 208 includes one or more nucleotides that are capable of base pairing with high reliability with a complementary nucleotide. Examples of such nucleotides include, but are not limited to, 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine, and 2-thio-uridine.

In certain embodiments, first oligonucleotide 208 can correspond to, or contain one or more fragments of, specialized nucleic acid species. For example, first oligonucleotide 208 can correspond to, or contain one or more fragments of, a locked nucleic acid (LNA), a peptide nucleic acid (PNA), an unlocked nucleic acid (UNA), and/or a morpholino oligomer.

The length of first oligonucleotide 208 (e.g., the number of nucleotides in first oligonucleotide 208) can generally be selected as desired to ensure efficient and selective hybridization with second oligonucleotide 212. In some embodiments, first oligonucleotide 208 can include at least 5 (e.g., at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100) nucleotides.

In some embodiments, first oligonucleotide 208 can have between 5-30, between 5-25, between 5-20, between 10-20, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides.

In certain embodiments, first oligonucleotide 208 can have no more than 5 (e.g., no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100) nucleotides.

In some embodiments, first oligonucleotide 208 can be fully single stranded. Alternatively, in certain embodiments, first oligonucleotide 208 can be at least partially double stranded. A partially double stranded region of first oligonucleotide 208 can be at the 3′ end of the oligonucleotide, at the 5′ end of the oligonucleotide, or between the 5′ end and 3′ end of the oligonucleotide.

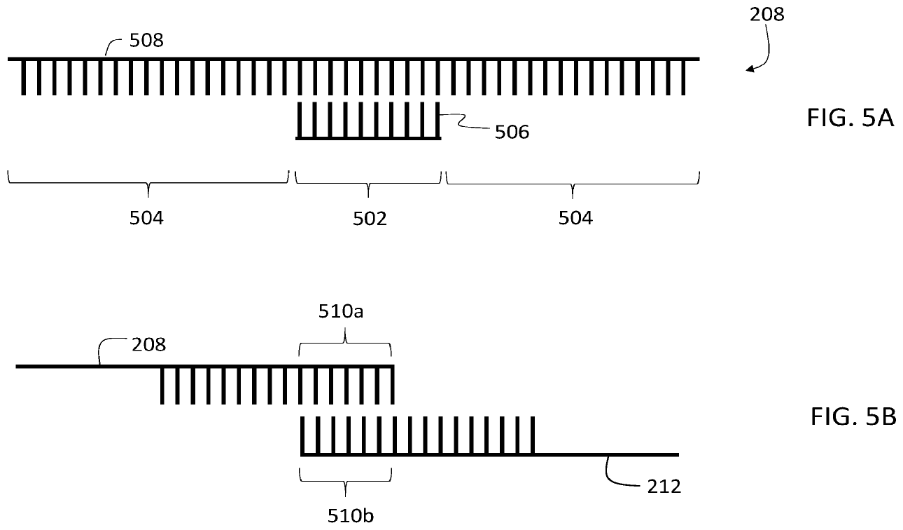

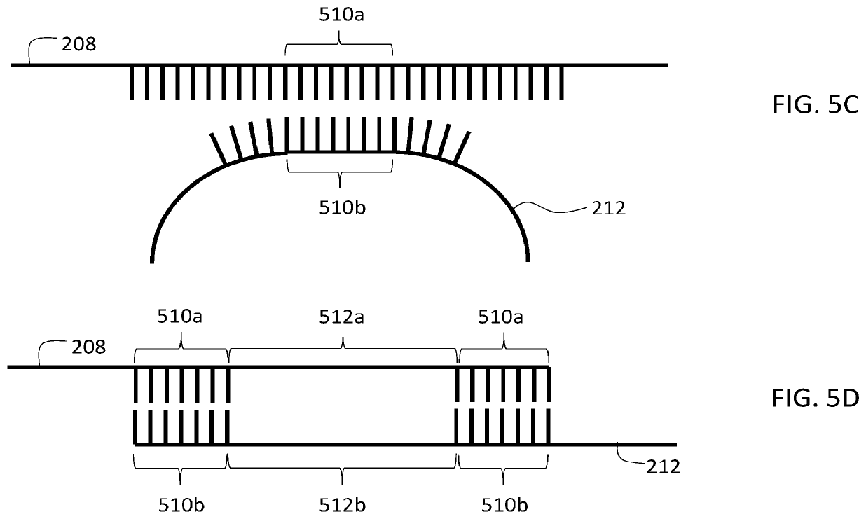

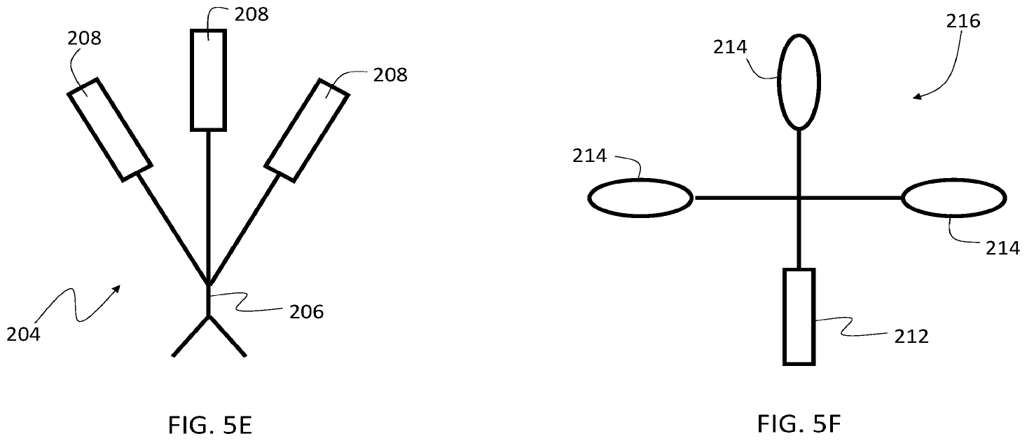

FIG. 5A is a schematic diagram of a first oligonucleotide 208 that includes two single stranded regions 504 and a double stranded region 502. As discussed above, double stranded region 502 can be positioned at the 3′ end of first oligonucleotide 208, and the 5′ end of first oligonucleotide 208, or at an intermediate position between the 3′ and 5′ ends. In certain embodiments, first oligonucleotide 208 can include more than one double stranded region (e.g., two or more, three or more, four or more, five or more, or even more double stranded regions).

The double stranded region can be formed by a secondary oligonucleotide strand 506 that is bound (e.g., hybridized) to a primary oligonucleotide strand 508 of first oligonucleotide 208, as shown in FIG. 5A. Alternatively, or in addition, first oligonucleotide 208 can include a secondary structure that allows folding of a single stranded first oligonucleotide 208. At least partial complementarity between different portions of the single strand allows the portions to hybridize, forming one or more double stranded regions from the single strand.

The one or more double stranded regions 502 of first oligonucleotide 208 can each, and collectively, extend over a percentage of the total length (e.g., the total number of nucleotides) in first oligonucleotide 208. In some embodiments, for example, one of more of the double stranded regions individually extends, or all of the double stranded regions collectively extend, over 1% or more (e.g., 5% or more, 10% or more, 15% or more, 20% or more, 25% or more, 30% or more, 35% or more, 40% or more, 50% or more) of the total length of first oligonucleotide 208.

In general, second oligonucleotide 212 can include any of the features described above for first oligonucleotide 208. Second oligonucleotide 212 can, in some embodiments, include the same number of nucleotides as first oligonucleotide 208. Alternatively, in certain embodiments, second oligonucleotide 212 can include a different number of nucleotides.

Second oligonucleotide 212 can have the same or different strand structure as first oligonucleotide 208. That is, second oligonucleotide 212 can be single stranded, double stranded, or partially double stranded, irrespective of the structure of first oligonucleotide 208. Second oligonucleotide 212 can generally include any number of double stranded regions, as described above for first oligonucleotide 208, extending over a portion of the total length of second oligonucleotide 212.

As discussed above, second oligonucleotide 212 hybridizes to first oligonucleotide 208 via base pairing so that first agent 204 and second agent 216 are co-localized in the sample at the location of target analyte 210. The efficiency of hybridization is related in part to the extent of complementarity between the sequences of the first and second oligonucleotides. As used herein, the percentage to which the sequences of the two sequences are complementary refers to the percentage of nucleotides in the shorter of the two sequences that have a complementary counterpart at a complementary location in the other sequence, such that the two counterparts pair during hybridization. In some embodiments, for example, the sequences of the two oligonucleotides are at least 70% (e.g., at least 75%, at least 80%, at least 85%, at least 90%, at least 95%, at least 99%) complementary.

As used herein, the term “at least partially complementary” means that two nucleotide sequences are sufficiently complementary that they hybridize. In general, two nucleotide sequences are at least partially complementary if their sequences are at least 50% complementary.

In general, second oligonucleotide 212 includes at least one binding region that hybridizes to a corresponding binding region of first oligonucleotide 208. The binding region can be located at the 3′ end, at the 5′ end, or intermediate between the two ends, of second oligonucleotide. Where second oligonucleotide 212 includes multiple binding regions, any of the binding regions can be located as above.

In some embodiments, the binding region of second oligonucleotide 212 is at least partially complementary to, and hybridizes with, the 3′ end of first oligonucleotide 208. In certain embodiments, the binding region of second oligonucleotide 212 is at least partially complementary to, and hybridizes with, the 5′ end of first oligonucleotide 208. FIG. 5B shows a schematic diagram of first and second oligonucleotides 208 and 212 with respective binding regions 510a and 510b. Binding region 510b of second oligonucleotide is at least partially complementary to a 3′ or 5′ end of first oligonucleotide 208 in FIG. 5B.

In certain embodiments, the binding region of 510b second oligonucleotide 212 is at least partially complementary to, and hybridizes with, an intermediate region of first oligonucleotide 208. FIG. 5C shows a schematic diagram in which binding region 510b of second oligonucleotide 212 binds with an intermediate binding region 510 of first oligonucleotide 208.

In some embodiments, the binding region 510b of second oligonucleotide 212 is at least partially complementary to, and hybridizes with, the entire first oligonucleotide 208. In certain embodiments, the binding region 510a of first oligonucleotide 208 is at least partially complementary to, and hybridizes with, the entire second oligonucleotide.

In certain embodiments, one or both of first and second oligonucleotides 208 and 212 includes multiple binding regions separated by one or more non-binding regions. FIG. 5D is a schematic diagram showing first and second oligonucleotides 208 and 212, each of which includes multiple binding regions 510a and 510b, respectively, separated by non-binding regions 512a and 512b, respectively. In general, each of the binding regions can have any of the properties discussed above in connection with first and second oligonucleotides 208 and 212 and their respective binding regions.

The non-binding regions 512a and 512b can be formed by a variety of different linking species, including non-complementary nucleotide sequences and spacer moieties that do not include nucleotides. Non-binding regions 512a-b can have the same or different geometric lengths, and binding regions 510a-b can have the same or different lengths (e.g., the same or different numbers of nucleotides). Within each oligonucleotide (e.g., 208 and/or 212), binding regions and non-binding regions can have the same or different lengths.

In some embodiments, binding species 206 can be conjugated to multiple first oligonucleotides 208 in the first agent. FIG. 5E is a schematic diagram showing a first agent 204 in which binding species 206 is conjugated to three first oligonucleotides 208. In general, each of the first oligonucleotides 208 has the same nucleotide sequence, so that the second oligonucleotide 212 can hybridize with any of the first oligonucleotides. While three first oligonucleotides are conjugated to binding species 206 in FIG. 5E, more generally 2 or more (e.g., 3 or more, 4 or more, 5 or more, 6 or more, 7 or more, 8 or more, or even more) first oligonucleotides can be conjugated to binding species 206. By conjugating more than one first oligonucleotide to binding species 206, additional reactive species can be selectively deposited in the sample at the location of target analyte 210, increasing the rate and amount of labeling species that can be deposited in the sample in proximity to that location.

In some embodiments, second oligonucleotide 212 of second agent 216 is conjugated to multiple reactive species 214. FIG. 5F is a schematic diagram showing a second agent 216 in which second oligonucleotide 212 is conjugated to three reactive species 214. The three reactive species can all be the same, or one or more can be different from the others. While three reactive species 214 are conjugated to second oligonucleotide 212 in FIG. 5F, more generally 2 or more (e.g., 3 or more, 4 or more, 5 or more, 6 or more, 7 or more, 8 or more, or even more) reactive species can be conjugated to second oligonucleotide 212. By conjugating more than reactive species to second oligonucleotide 212, additional reactive species can be selectively deposited in the sample at the location of target analyte 210, increasing the rate and amount of labeling species that can be deposited in the sample in proximity to that location.

### Reactive Species and Labeling Species

As discussed above, in second agent 216, second oligonucleotide 212 is conjugated to a reactive species 214 that reacts with labeling species 218. Reactive species 214 can correspond to any one or more of a variety of different chemical or biochemical species and moieties. In some embodiments, for example, reactive species 214 corresponds to a catalytic agent that catalyzes a reaction of labeling species 218. Examples of catalytic agents that can correspond to reactive species 214 include, but are not limited to, enzymes, transition metal-based organometallic moieties, peroxide containing moieties, and photoactivatable species. Examples of suitable enzymes include, but are not limited to, horseradish peroxidase (HRP) and soybean peroxidase. In some embodiments, reactive species 214 can include a hemin-containing complex which can mimic HRP, such as hematin.

In general, labeling species 218 includes at least one labeling moiety. A variety of different labeling moieties can be used, depending upon the nature of the methodology used to identify and quantify targets 210 in sample 202. In some embodiments, for example, labeling species 218 includes a dye. As used herein, a “dye” is a moiety that interacts with incident light, and from which emitted light can be measured and used to detect the presence of the dye in a sample. In general, a dye can be a fluorescent moiety, an absorptive moiety (e.g., a chromogenic moiety), or another type of moiety that emits light, or modifies incident light passing through or reflected from a sample where the dye is present so that the presence of the dye can be determined by measuring changes in transmitted or reflected light from the sample.

In certain embodiments, the labeling moiety can include a hapten. The hapten can subsequently (or concurrently) be bound to a dye moiety to provide a labeling moiety that can be detected by measuring emitted, transmitted, or reflected light from the sample.

When the labeling moiety of labeling species 218 includes a dye, a wide variety of different dyes can be used. For example, the dye can be a xanthene-based dye, such as a fluorescein dye and/or a rhodamine dye. Examples of suitable fluorescein and rhodamine dyes include, but are not limited to, fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′, 5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110.

The dye can also be a cyanine-based dye. Suitable examples of such dyes include, but are not limited to, the dyes Cy3, Cy5 and Cy7. The dye can also be a coumarin dye (e.g., umbelliferone), a benzimide dye (e.g., any of the Hoechst dyes such as Hoechst 33258), a phenanthridine dye (e.g., Texas Red), an ethidium dyes, an acridine dyes, a carbazole dye, a phenoxazine dye, a porphyrin dye, a polymethine dye (e.g., any of the BODIPY dyes), and a quinoline dye.

When the dye is a fluorescent moiety, the dye can be a moiety corresponding to any of the following non-limiting examples and/or derivatives thereof: pyrenes, coumarins, diethylaminocoumarins, FAM, fluorescein chlorotriazinyl, fluorescein, Rl 10, JOE, R6G, tetramethylrhodamine, TAMRA, lissamine, naphthofluorescein, Texas Red, Cy3, and Cy5.

In certain embodiments, the dye can include one or more quantum dot-based species. Quantum dot-based fluorophores are available with fluorescence emission spectra in many different spectral bands, and suitable quantum dot-based dyes can be used as labeling species in the methods described herein.

### Tyramide Signal Amplification

In some embodiments, reactive species 214 is an enzyme that participates an enzyme-mediated reaction to deposit labeling species 218 (or a derivative thereof) at locations in the sample that are proximate to second agent 216 and, therefore, to target analyte 210. As an example of enzyme-mediated deposition of a labeling species, reactive species 214 can be horseradish peroxidase (HRP) or another species that mimics the activity of HRP. HRP can be used in the methods described herein as a catalytic agent for tyramide signal amplification (TSA).

To implement TSA, labeling species 218 includes a labeling moiety (such as a dye, as described above) conjugated to a tyramide species. When sample 202 is initially contacted with labeling species 218, the tyramide species is in an inactive form. However, HRP catalyzes conversion of the tyramide species to an active form that is capable of binding with sample 202. Following conversion of the tyramide species to its active form, the labeling moiety binds to sample 202 at locations proximate to where it is generated (e.g., at the location of second agent 216 and target analyte 210). FIG. 2C illustrates schematically the deposition of labeling species 218 (which can include an active tyramide species conjugated to a labeling moiety) at location 222, in proximity to target analyte 210.

By adjusting the amount of tyramide-containing labeling species 218 introduced into sample 202 and the amount of time during which the enzyme-mediated activation process continues, the amount of labeling species 218 deposited in sample 202 can be controlled. As a result, the signal that is detected and that corresponds to labeling species 218 (and therefore, to target analyte 210) can be “amplified”. In the context of the present disclosure, amplification refers to the linking of more than one labeling species 218 to each target analyte 210. With reference to immunohistochemical labeling methods in which each binding antibody is conjugated to a single labeling species (e.g., a single fluorophore moiety), the TSA technique can be used to deposit multiple labeling species 218 (or derivatives thereof) in the sample to generate measurable signals corresponding to a single target analyte 210, thereby increasing the amplitude or intensity of measured signals corresponding to the single target analyte relative to signals that would otherwise be measured from a single labeling species.

In general, a ratio of the number of labeling species 218 that can be deposited in the sample in proximity to a single target analyte 210 is increased beyond 1:1 by implementing the TSA methodology described above. In some embodiments, for example, the ratio is 2:1 or more (e.g., 3:1 or more, 4:1 or more, 5:1 or more, 6:1 or more, 8:1 or more, 10:1 or more, 20:1 or more, 30:1 or more, or even more).

Amplification provides a number of important advantages. First, because measurable signals corresponding to target analytes are of higher amplitude or intensity than in the absence of amplification, exposure times and measurement times can be reduced. Second, due to the increased amplitude or intensity of measurement signals, target analytes that are present in the sample at relatively low concentrations—and whose corresponding measurement signals would otherwise be relatively weak absent amplification—can be detected with greater reliability. Third, due to the increased amplitude or intensity of measurement signals, compensation for the confounding effects of tissue autofluorescence is easier, as is the detection of the measurement signals against a background autofluorescence signal that might otherwise obscure some or all of the measurement signals.

Amplification can also be used to adjust measurement signals corresponding to different target analytes. For example, in samples where certain analytes are present at significantly smaller concentrations than other analytes, the amplitude or intensity of measurement signals corresponding to low-concentration analytes can be amplified so that they more closely match the amplitudes or intensities of signals corresponding to other target analytes in the sample that are present in higher concentrations. In this manner, the range amplitudes or intensities of the measurement signals can be reduced, so that the dynamic range of the measurement system used to detect the measurement signals can also be smaller relative to the dynamic range that would otherwise be used to measurement signals in the absence of amplification.

Further, the presence of low-concentration analytes in a sample (such as very weakly expressed biomarkers) can be visualized along with higher-concentration analytes for co-expression analysis, protein regulation assessments, and other comparative analyses that would be more challenging if both low- and higher-concentration analytes were not detected and visualized simultaneously.

When the reactive species 214 corresponds to an enzyme or other catalytic agent, the enzyme or catalytic agent can mediate the deposition of labeling species 218 in the sample via any of a variety of different types of reactions. In some embodiments, for example (such as TSA with HRP-mediated deposition of a tyramide-conjugated labeling moiety), the reaction which is mediated by the enzyme or catalytic agent is an oxidation-reduction reaction. Other examples of suitable enzyme or catalytic agent-mediated reactions include, but are not limited to, deprotonations, eliminations, radical generation reactions, deprotections, and rearrangements.

For oxidation-reduction reactions (such as TSA with HRP-mediated deposition of labeling species 218), a variety of different oxidation and/or reduction agents can be used. In some embodiments, for example, the oxidation agent is H₂O₂. A variety of other agents can also be used.

Further, it should also be noted that while in some embodiments, deposition of labeling species 218 (or a derivative thereof) in sample 202 is irreversible, in certain embodiments the deposition of labeling species 218 in sample 202 is reversible, and labeling species 218 can be removed from sample 202 following deposition by methods such as washing, one or more chemical reactions to liberate labeling species 218, and physical methods such as heating and exposure to radiation (e.g., photocleavage or photoionization or sputtering) of the labeling species 218.

Labeling species 218 can be stably bound to sample 202 at room temperature for 48 hours or more. In some embodiments, a sample labeled with labeling moiety 218 refrigerated at 4° C. can be stable for up to at least 4 weeks, and a sample frozen at −20° C. or −80° C. can remain stably bound to labeling species 218 for up to 4 months or more.

The stability of the binding between labeling species 218 and sample 202 can vary depending upon the nature of both the labeling species 218 and the sample 202. In general, the binding is stable for at least 48 hours when sample 202 is stored relatively close to room temperature or below room temperature. For example, in certain embodiments, the binding is stable for at least 48 hours when sample 202 is maintained within about 5° C. of room temperature. In certain embodiments, the binding is stable for at least 48 hours when the sample 202 is maintained at a temperature between 0° C. and 40° C. (e.g., between 10° C. and 40° C., between 15° C. and 40° C., between 20° C. and 40° C., between 25° C. and 40° C., between 30° C. and 40° C., between 35° C. and 40° C., between 0° C. and 35° C., between 5° C. and 35° C., between 10° C. and 35° C., between 15° C. and 35° C., between 20° C. and 35° C., between 25° C. and 35° C., between 30° C. and 35° C., between 0° C. and 30° C., between 5° C. and 30° C., between 10° C. and 30° C., between 15° C. and 30° C., between 20° C. and 30° C., between 25° C. and 30° C., between 0° C. and 25° C., between 5° C. and 25° C., between 10° C. and 25° C., between 15° C. and 25° C., between 20° C. and 25° C., between 0° C. and 20° C., between 5° C. and 20° C., between 10° C. and 20° C., between 15° C. and 20° C., between 0° C. and 15° C., between 5° C. and 15° C., between 10° C. and 15° C., between 0° C. and 10° C., between 5° C. and 10° C., and between 0° C. and 5° C.).

Additional methods and aspects of TSA are described, for example, in Faget et al., Methods Mol. Biol. 1318: 161-72 (2015), the entire contents of which are incorporated herein by reference.

### Multiplexed Analysis

Referring again to FIGS. 1 and 2C, after labeling species 218 has been detected in the sample, second agent 216 can optionally be removed from the sample. In particular, because first oligonucleotide 208 and second oligonucleotide 212 are hybridized, removal of second agent 216 involves de-hybridizing the first and second oligonucleotides. As discussed above, dehybridization can generally be accomplished under significantly milder conditions than antibody removal from a sample, which occurs in certain immunohistochemical methods.

De-hybridization can also be used to control the amount of labeling species 218 that is deposited in sample 202 (i.e., during amplification). More particularly, de-hybridization of the first and second oligonucleotides can be used to terminate the reaction (e.g., a catalytic reaction such as enzyme-mediated deposition of labeling species 218) between reactive species 214 and labeling species 218, thereby controlling the amount of time during which deposition of labeling species 218 in the sample occurs.

Various methods can be used to achieve de-hybridization of the first and second oligonucleotides. In some embodiments, for example, de-hybridization of the oligonucleotides can be achieved by exposing the oligonucleotides to one or more chaotropic reagents, such as dimethyl sulfoxide (DMSO) and formamide, wherein the molar concentration of the chaotropic reagent in a solution thereof is 60% or more (e.g., 70% or more, 80% or more, 90% or more). Alternatively, de-hybridization can be performed by washing sample 202, by heating sample 202, and by combinations of the foregoing techniques.

De-hybridization of the first and second oligonucleotides in sample 202, followed by a washing step to remove free second agent 216 following de-hybridization, yields a sample 202 in which first agent 204 remains bound to target analyte 210 through binding species 206, and labeling species 218 remains bound to sample 202 in proximity to target analyte 210. In effect, de-hybridization returns sample 202 to a state similar to that shown in FIG. 2A, with the added presence of labeling species 218. FIG. 2D shows sample 202 in schematic form following de-hybridization of the first and second oligonucleotides and subsequent removal of free second agent 216 from the sample.

Some or all of the steps shown in flow chart 100 can optionally be repeated to selectively identify and quantify a second (and subsequent) target analytes 210 in sample 202. Specifically, sample 202 can be contacted with another first agent that includes a binding species 206 that selectively binds to a different target analyte 210 in sample 202 and a first oligonucleotide that is different from the first oligonucleotide of the previous first agent. Then, the sample can be contacted with another second agent that includes a reactive species (e.g., any of the reactive species described above) and a second oligonucleotide that is at least partially complementary to, and hybridizes with, the first oligonucleotide of the newly added first agent.

Following addition of the additional second agent, a new labeling species can be introduced that reacts with the reactive species to deposit the new labeling species (or a derivative thereof) at a location in the sample proximate to the newly added first and second agents, and therefore, proximate to the second target analyte 210 to which the newly added first agent selectively binds. Measured signals corresponding to the newly added labeling species can be used to identify and quantify the second target analyte 210 in the sample.

The newly added second agent can again be removed from the sample via de-hybridization of the first and second oligonucleotides as described above, and additional cycles can be repetitively performed to selectively identify and quantify multiple different target analytes 210 in sample 202.

Each repetition of some or all of the steps of flow chart 100 is referred to as analytical cycle, and in general, any number of cycles can be performed to selectively identify and quantify different target analytes 210 in sample 202. In some embodiments, for example, the number of such cycles is N, where N is 2 or more (e.g., 3 or more, 4 or more, 5 or more, 6 or more, 7 or more, 8 or more, 10 or more, 12 or more, 15 or more, or even more).

In general, for each different combination of first and second agents targeting a different target analyte 210 in the sample for analysis, a different labeling species 218 is selectively deposited in the sample proximate to the particular target analyte 210. By selecting different labeling species, different target analytes can selectively be interrogated by isolating and optionally quantifying contributions to measured emitted, reflected, or transmitted light from the sample that arise specifically from the different labeling species. Since each species is effectively “mapped” to a different target analyte, identification and quantification of specific analytes can be achieved by isolating measurement signals corresponding to their associated labeling species.

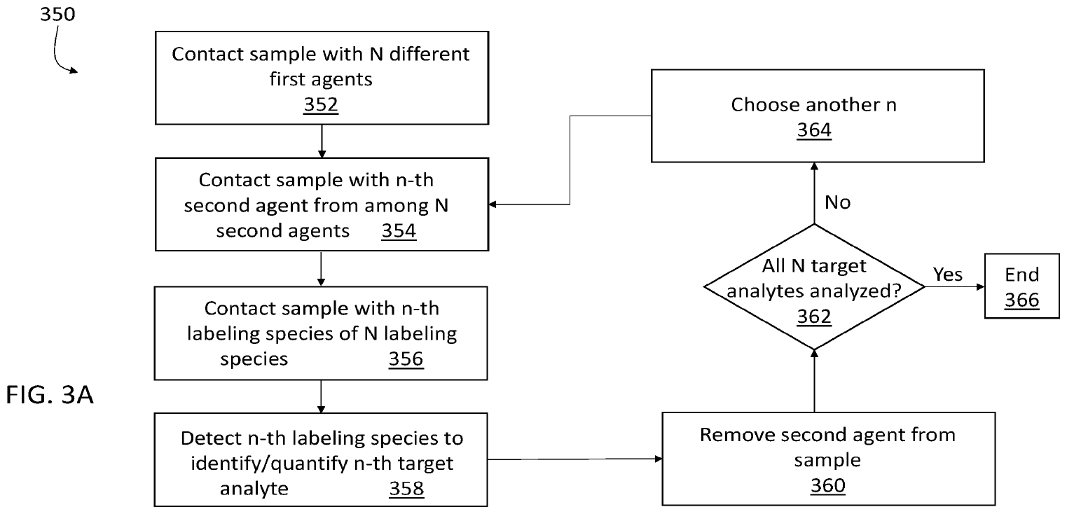

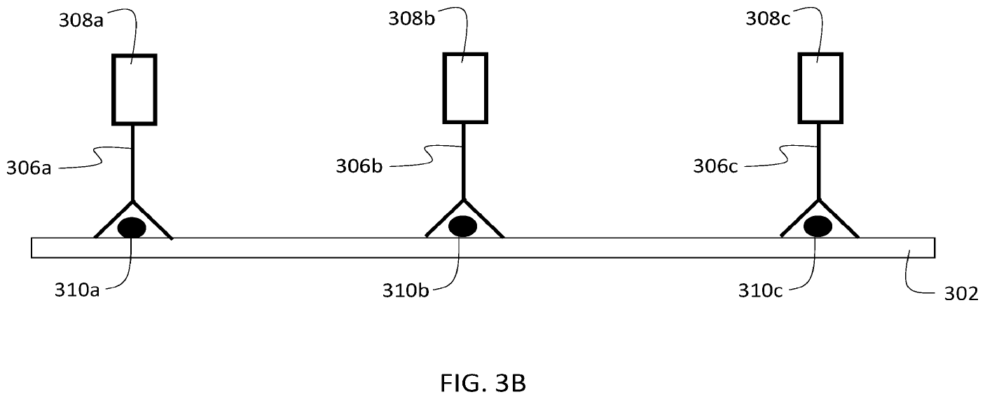

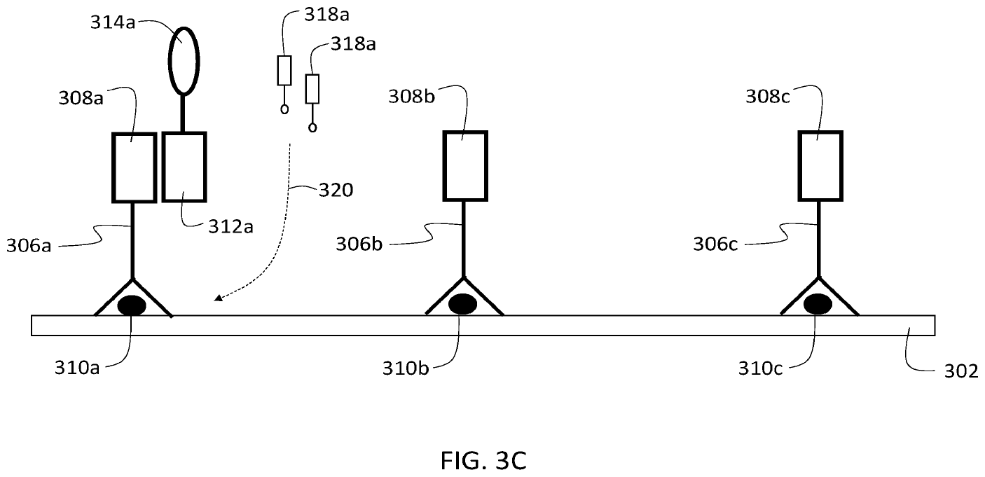

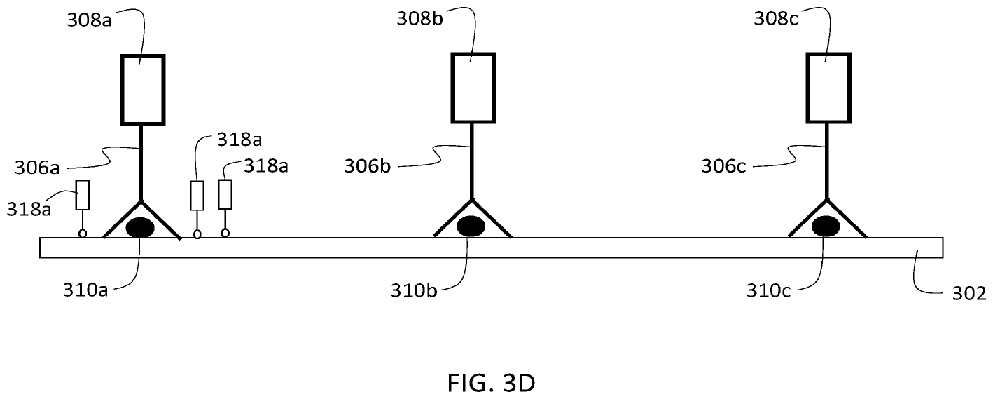

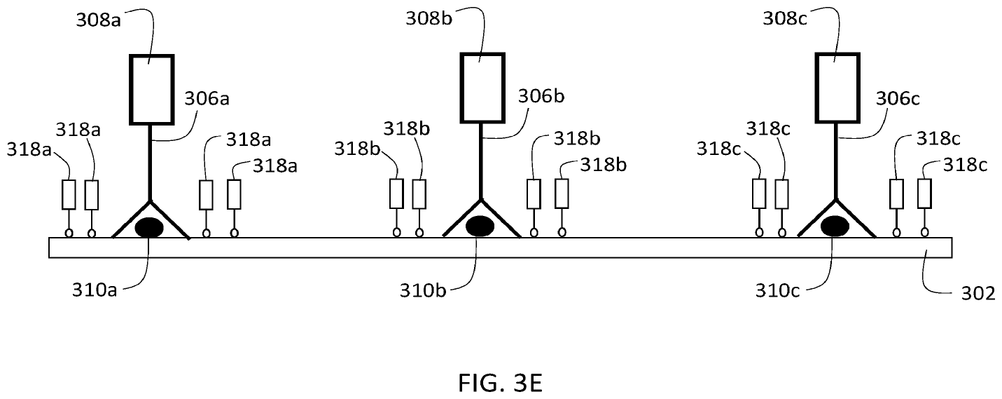

Multiple target analytes 210 can also be analyzed by multiplexing the addition of different first agents and second agents to the sample. FIG. 3A is a flow chart 350 showing a series of example steps for analyzing N different target analytes in a sample. In general, N can be 2 or more (e.g., 3 or more, 4 or more, 5 or more, 6 or more, 7 or more, 8 or more, 10 or more, 12 or more, 15 or more, or even more).

In a first step 352, the sample is contacted with N different first agents. Each of the N different first agents includes a binding species that specifically binds to one of the N different target analytes, and a unique first oligonucleotide conjugated to the binding agent. In other words, the binding agent and conjugated first oligonucleotide of each of the N first agents are different from the binding agents and conjugated first oligonucleotide of the other first agents among the N first agents.

Next, in step 354, one of the n target analytes is selected for analysis, and a second agent that includes a reactive species conjugated to a second oligonucleotide that is at least partially complementary to, and hybridizes to, the first oligonucleotide of the first agent that selectively binds the n-th target analysis is contacted to the sample. The second oligonucleotide is thus bound to the sample at locations corresponding to the n-th target analyte (and the corresponding n-th first agent).

Then, in step 356, the sample is contact with an n-th labeling species that includes a labeling moiety that is different from the labeling moieties of the other (n−1) labeling species. The labeling species reacts with the reactive species of the n-th second agent, depositing the n-th labeling species in the sample in proximity to the n-th target analyte.

Next, in step 360, the n-th second agent is removed from the sample by de-hybridization and washing as described previously. In step 362, if all N target analytes have been analyzed, the procedure terminates at step 366. If not, another n-th analyte among the N target analytes is selected for analysis, and the procedure returns to step 354.

The foregoing procedure is illustrated schematically in FIGS. 3A-3E for a sample that includes N=3 target analytes for analysis. FIG. 3B is a schematic diagram showing a sample 302 with three different target analytes 310a-c. In a first step of the analysis of sample 302, three different first agents are contacted to the sample, each with a different binding species 306a-c that specifically binds to a different one of the three target analytes 310a-c. The first agents each include a different first oligonucleotide 308a-c. As shown in FIG. 3A, contacting each of the first agents to the sample yields a sample in which the first agents are selectively bound only to the corresponding target analytes 310a-c to which their binding species 306a-c are matched.

In a subsequent step of the analysis, a second agent that includes a reactive species 314a and a second oligonucleotide 312a that is at least partially complementary to first oligonucleotide 308a is contacted to the sample. As shown in FIG. 3C, second oligonucleotide 312a hybridizes to first oligonucleotide 308a, selectively binding this second agent to the first agent bound to target analyte 310a. The second agent does not bind to either of the first agents bound to target analytes 310b and 310c, owing to a lack of complementarity between second oligonucleotide 312a and first oligonucleotides 308b and 308c.

Then, as shown in FIG. 3C, a labeling agent 318a is contacted to the sample, and reacts with reactive agent 314a in the manner described above to selectively deposit labeling agent 318a (or a derivative thereof) in proximity to target analyte 310a. The reaction and deposition of labeling agent 318a is illustrated schematically in FIG. 3C by dashed arrow 320a.

After deposition of labeling agent 318a, the second agent (i.e., second oligonucleotide 312a and reactive agent 314a) is removed from sample 302 by de-hybridization and washing, as described above. The resulting sample 302 is shown schematically in FIG. 3D. Each of the different first agents remains bound to corresponding different target analytes 310a, and the labeling agent 318a deposited in proximity to target analyte 310a also remains bound to sample 302.

The cycle illustrated in FIG. 3C and described in steps 354-360 of flow chart 350 is then repeated, first with a second agent that includes a second oligonucleotide that selectively hybridizes—through at least partial complementarity—with first oligonucleotide 308b of the first agent bound to target analyte 310b, and then with a second agent that includes a second oligonucleotide that selectively hybridizes, through at least partial complementarity, with first oligonucleotide 308c of the first agent bound to target analyte 310c. In each cycle, a different labeling agent contacts the sample and is deposited in proximity to the corresponding target analyte.

After three complete labeling cycles, sample 302 appears as shown schematically in FIG. 3E, with each of the three different first agents remaining bound to their corresponding target analytes 310a-c, and three different labeling agents 318a-c deposited, respectively, in proximity to each of the three different target analytes 310a-c.

In general, each of the different labeling agents 318a-c can correspond to any of the labeling agents described above. To implement analysis of multiple target analytes in a sample, the labeling agents are generally selected such that they generate different measurement signals. For example, in embodiments where each of the labeling agents include a fluorescent or chromogenic dye moiety, the agents are selected so that each of the dye moieties have different spectral properties (e.g., absorption, emission), so that measured light emitted from, transmitted through, or reflected from the sample can separated into contributions from each of the dyes, and used to separately identify and quantify each of the target analytes 310a-c in the sample.

### Reporting Agents

Reporting agents can be used in connection with the methods described above. In general, a reporting agent includes an oligonucleotide conjugated directly or indirectly to a labeling moiety. Reporting agents that are used in the methods described herein include oligonucleotides that are at least partially complementary to, and hybridize to, corresponding first oligonucleotides of the first agents described above. Accordingly, the oligonucleotides of the reporting agents can generally include any of the features of the first and second oligonucleotides discussed previously. Further, the reporting agents can include any one or more of the different types of labeling moieties described above.

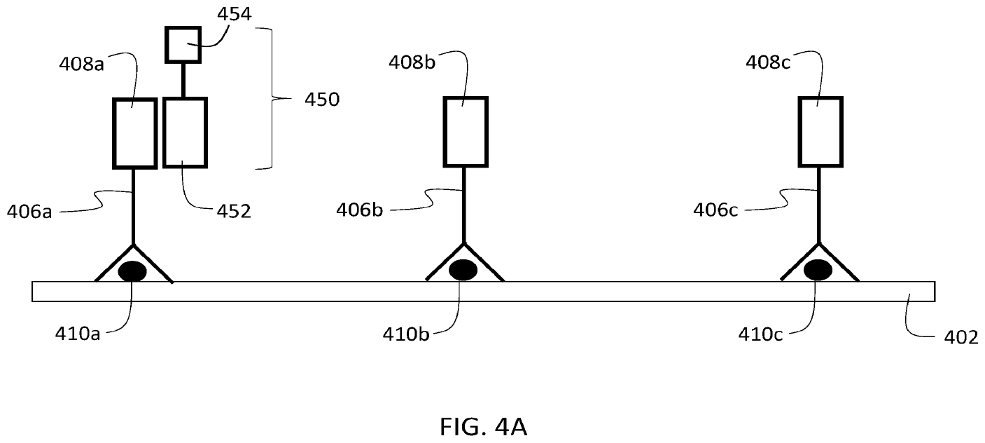

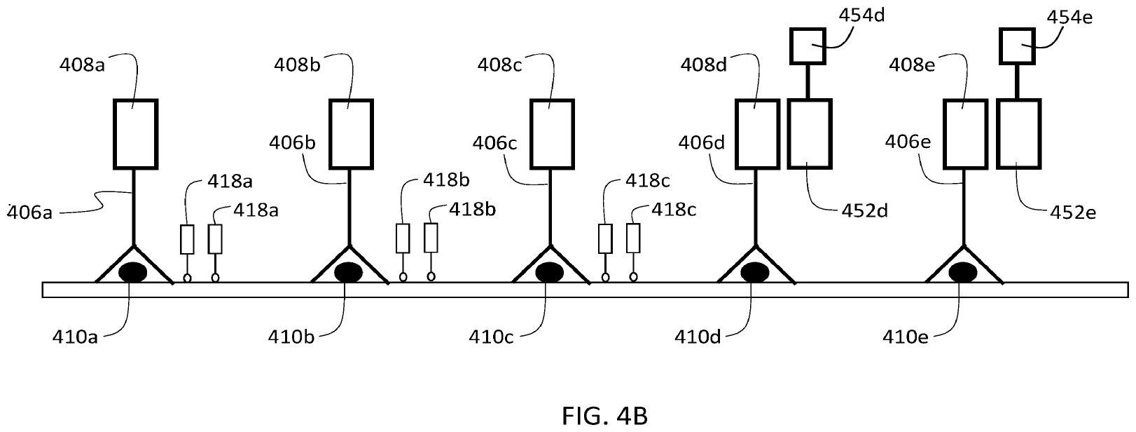

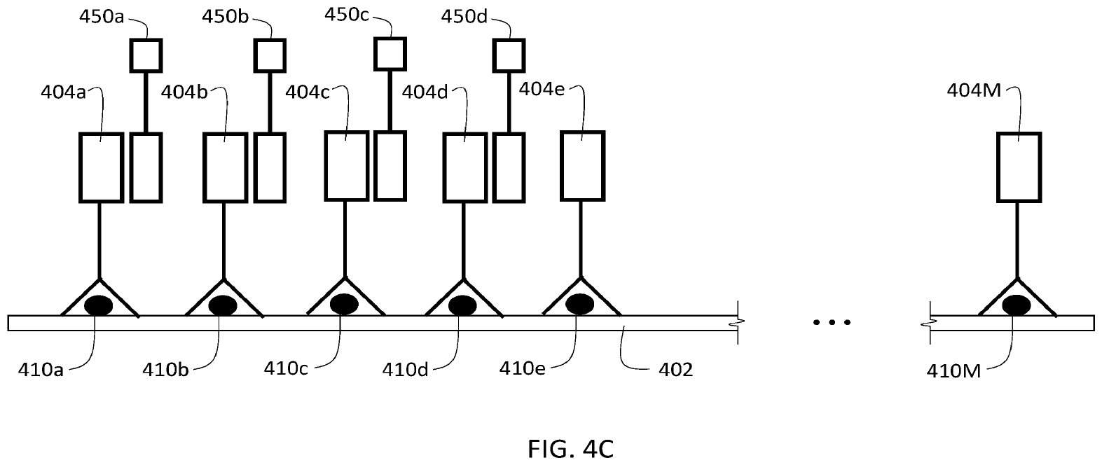

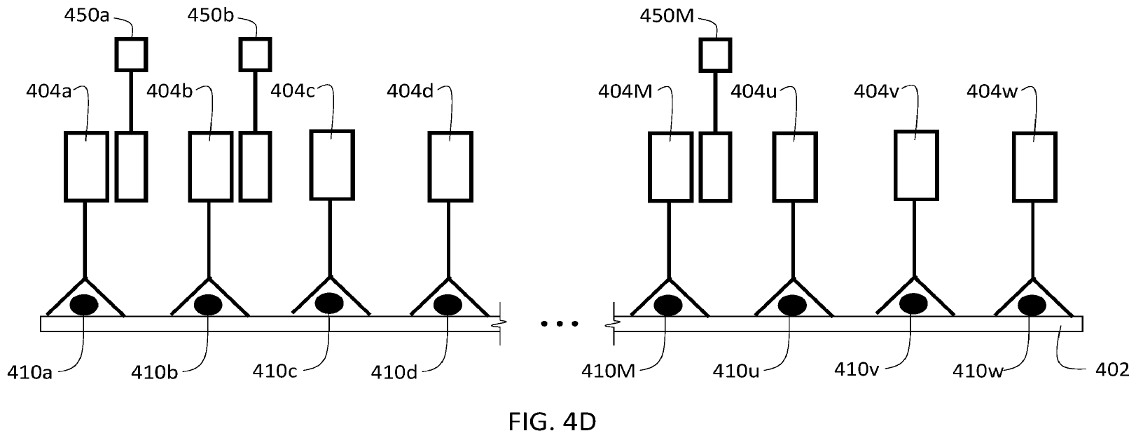

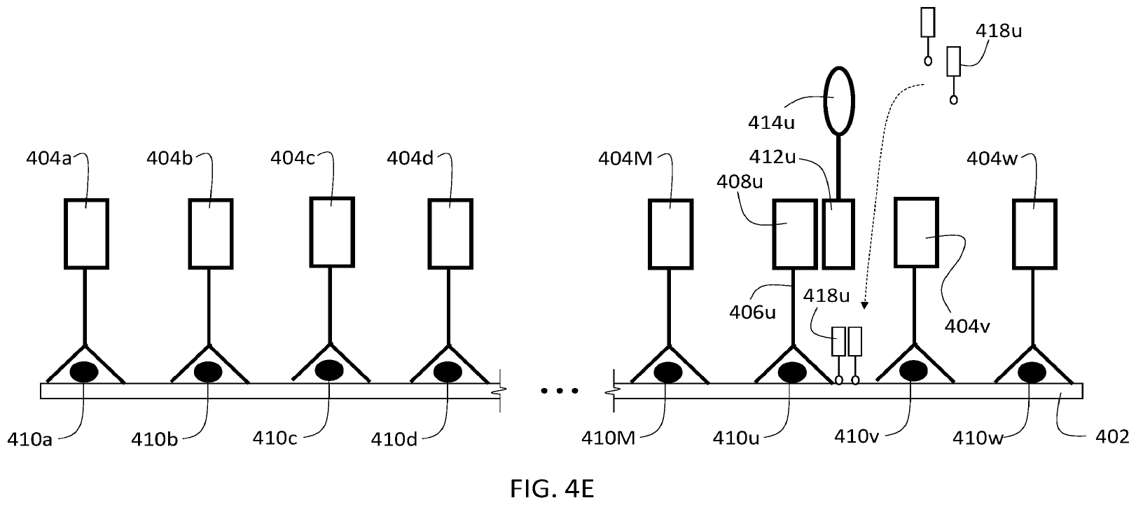

The use of reporting agents can be integrated into the procedures described herein at various points, and to achieve a variety of objectives. For example, in some embodiments, reporting agents can be used to verify the presence of particular target analytes in a sample. FIG. 4A shows a schematic diagram of a sample 402 that includes three different target analytes 410a-c, analogous to sample 302 in FIG. 3A. Three different first agents are selectively bound to sample 302, one each to each of the different target analytes. Each of the first agents includes a binding agent 406a-c, and a first oligonucleotide 408a-c.

Prior to introducing a second agent into sample 402 (e.g., as in step 354 of flow chart 350), the sample is contacted with a reporting agent 450 to verify the presence of target analyte 410a in sample 402. As shown in FIG. 4A, reporting agent 450 includes a labeling moiety 454 conjugated to an oligonucleotide 452. Oligonucleotide 452 is at least partially complementary to oligonucleotide 408a, and hybridizes to oligonucleotide 408a when reporting agent 450 is introduced into the sample. The nucleotide sequence of oligonucleotide 452 is sufficiently non-complementary to the sequences of first oligonucleotides 408b and 408c such that oligonucleotide 452 does not hybridize to either of these first oligonucleotides. After washing away unbound reporting agent 450, the remaining reporting agent 450 in sample 402 is selectively bound to the first agent that binds target analyte 410a.

The presence of target analyte 410a in sample 402 can be verified by measuring light emitted from, reflected from, or transmitted through, sample 402. In particular, by detecting a portion of the measured light that corresponds to labeling moiety 454, the presence of target analyte 410a in sample 402 can be verified. Optionally, the amount of target analyte 410a in sample 402 can also be quantified based on the measured signal contributions that are attributable to labeling moiety 454.

The locations in sample 402 where target analyte 410a is present can be used to determine a region of interest for further measurements and/or delivery of agents to sample 402. For example, if target analyte 410a is localized to only a portion of sample 402, subsequent sample light emission, reflection, or transmission measurements, and/or delivery of agents to sample 402, can optionally be restricted to only that region of the sample.

Alternatively, when target analyte 410a is not present in the sample, the first agent that selectively binds to target analyte 410a (i.e., with binding species 406a and first oligonucleotide 408a) will not bind to sample 402. As a consequence, reporting agent 450 also will not bind to sample 402 due to the absence of first oligonucleotide 408a, and measured light from sample 402 will not include contributions from labeling moiety 454, indicating the absence of target analyte 410a in the sample. As a result, the steps of contacting the sample with a second agent and a labeling species to specifically deposit a labeling moiety in proximity to target analyte 410a can be omitted.

In the foregoing example, a single reporting agent 450 is used to selectively identify and optionally quantify a single target analyte 410a in a sample. However, similar labeling and measurements can be performed with multiple different reporting agents—each of which includes a different oligonucleotide that selectively hybridizes only to one of first oligonucleotides 408a-c in FIG. 4A, and a different labeling moiety, such that each reporting agent 450 targets a specific target analyte 410a-c. Each of the reporting agents can be contacted to the sample, measured, and removed serially, or alternatively, groups of multiple reporting agents can be applied to the sample. Provided that the measured emitted, transmitted, or reflected light from the sample can be decomposed into contributions from the labeling moieties of each of the individual reporting agents, parallel analysis of 2 or more (e.g., 3 or more, 4 or more, 5 or more, 6 or more, 8 or more, 10 or more, or even more) different target analytes in the sample can be performed.

Reporting agents can also generally be used at any intermediate step in the analytical procedures described herein. For example, referring to the procedure described in connection with FIGS. 3A-3E, reporting agents can be used to verify the presence of any of target analytes 310a-c (and corresponding target analytes 410a-c in FIG. 4A). They can be introduced at any stage of the procedure following contact of the sample with one or more first agents, as described above.

In some embodiments, reporting agents can be used together with, or as alternatives to, labeling species to identify and quantify target analytes in a sample. FIG. 4B shows a schematic diagram of a sample 402 that includes five different target analytes 410a-e. The sample has been contacted with five different first agents, each with a different binding species 406a-e and a different first oligonucleotide 408a-e, selectively binding each of the five different first agents to one of the five different target analytes.

Three analysis cycles have also been performed on sample 402 in FIG. 4B to deposit labeling moieties in proximity to three different target analytes, as generally described above. As a result, three different labeling species 418a-c have been deposited in the sample in proximity to respective target analytes 410a-c.

Further, two different reporting agents have been introduced into the sample. A first one of the reporting agents includes an oligonucleotide 452d and a labeling moiety 454d. Oligonucleotide 452d is at least partially complementary to first oligonucleotide 408d, and hybridizes to first oligonucleotide 408d. Oligonucleotide 452d is not sufficiently complementary to any of the other first oligonucleotides in the sample to hybridize significantly to the other first oligonucleotides. Similarly, oligonucleotide 452e of the second reporting agent is at least partially complementary to first oligonucleotide 408e and hybridizes it first oligonucleotide 408e. Oligonucleotide 452e is not sufficiently complementary to any of the other first oligonucleotides in sample 402.

The labeling moieties in the labeling species 418a-c and the reporting agents (i.e., labeling species 454d-e) are selected such that each labeling species is different, and therefore has different spectral absorption, reflection, or emission properties. By analyzing emitted, transmitted, or reflected light from sample 402, the contributions from each of the different labeling moieties can be distinguished and selectively attributed to target analytes 410a-e. In this manner, each of the target analytes 410a-e in sample 402 can be identified and quantified.

Sample 402 in FIG. 4B includes a mixture of labeling moieties from labeling species and reporting agents. In some embodiments, however, reporting agents can be used to label and detect signals corresponding to all of the target analytes in a biological sample.

For example, FIG. 4C is a schematic diagram of a sample 402 that includes M different target analytes 410a . . . 410M. M can generally be 2 or more (e.g., 3 or more, 4 or more, 5 or more, 6 or more, 8 or more, 10 or more, 15 or more, 20 or more, 30 or more, 40 or more, 50 or more, or even more). The sample is contacted with a set of first agents 404a . . . 404M to selectively bind each one of the first agents to a different one of the M target analytes, as described above.

Then, reporting agents 450a . . . 450M are selectively hybridized to the first agents to label the M target analytes. Each reporting agent includes a different labeling moiety so that each of the M target analytes is labeled with a different labeling moiety. In some embodiments, the reporting agents are applied singly (i.e., serially) to sample 402, and light emitted, reflected, or transmitted from the sample with only one of the reporting agents present at a time is measured to identify and quantify the target analyte corresponding to the one reporting agent.

In certain embodiments, however, the sample is contacted with groups of reporting agents (e.g., groups of 2 or more reporting agents, groups of 3 or more reporting agents, groups of 4 or more, reporting agents, groups of 5 or more reporting agents, groups of 6 or more reporting agents, groups of 8 or more reporting agents, or groups of even more reporting agents), with each reporting agent selectively hybridizing to only one of the first agents bound to one of the target analytes in the sample. Light emitted, reflected, or transmitted from the sample with the multiple reporting agents of a group present in the sample is measured and decomposed into contributions from each of the labeling moieties of the reporting agents. These contributions can then be used to identify and quantify each of the target analytes corresponding to the reporting agents of the group.

The group of reporting agents can then be removed from the sample via de-hybridization as discussed previously, and a new group of reporting agents that selectively hybridizes to a different set of first agents bound to a different set of target analytes is introduced. The new group can include the same number of reporting agents or a different number of reporting agents relative to the prior group.

Analysis of the target analytes in the sample continues in this manner, with successive groups of reporting agents hybridized, detected, and removed, until all M target analytes have been analyzed.

In some embodiments, a hybrid analytical workflow can be implemented that uses both reporting agents and deposition of labeling moieties (e.g., via TSA) to analyze target analytes. Such procedures can be used, for example, for samples that include a relatively large number of target analytes, a few of which (e.g., between 1 and 8 of which) are particularly important. Hybrid procedures are also useful for samples that include a relatively large number of target analytes, and a few of the target analytes are expressed weakly or otherwise generate signals following labeling that are difficult to measure. Amplification of measurement signals corresponding to particularly important or weakly expressed target analytes can be used to achieve highly multiplexed sample characterization with particular attention to certain analytes.

An example of such a sample 402 is shown in FIG. 4D. The sample includes target analytes 410a . . . 410M and 410u, 410v, and 410w. Among these target analytes, 410u-410w are of particular importance.

One example of a hybrid analytical workflow is as follows. Sample 402 is contacted with first agents 404a . . . 404M and 404u-404w. Each of these first agents includes a different binding group that selectively binds to one of the target analytes, and a different first oligonucleotide.

After the first agents have been selectively bound and localized in sample 402 at locations corresponding to their respective target analytes, target analytes 410a . . . 410M are analyzed using reporting agents 450a . . . 450M in the manner described above in connection with FIG. 4C. Specifically, reporting agents are hybridized in groups or individually to the first agents in sample 402. Each reporting agent includes a different oligonucleotide that is at least partially complementary to only one of the first oligonucleotides of the first agents 404a . . . 404M, and a different labeling moiety. Reporting agents 450a . . . 450M can be hybridized to the sample one-at-a-time (e.g., serially), or in groups of 2 or more. To detect the reporting agents following hybridization, light emitted from, reflected from, or transmitted from the sample is measured and, if it includes contributions from multiple labeling moieties, decomposed into contributions from each of the labeling moieties. Target analytes corresponding to the reporting agents of each group are identified and optionally quantified, and then the group of reporting agents is removed from sample 402 by de-hybridization and washing. Analysis of the remaining target analytes 410a . . . 410M is performed in similar fashion by hybridizing groups of one or more selective reporting agents to corresponding first agents 404a . . . 404M, measuring signals corresponding to the reporting agents, and then removing the reporting agents in preparation for another analysis cycle.

Next, each of the high important target analytes 410u-410w is analyzed in turn. As shown schematically in FIG. 4E, the sample is contacted with a second agent that includes a second oligonucleotide 412u that selectively hybridizes to first oligonucleotide 408u. Conjugated to second oligonucleotide 412u is a reactive species 414u. Following hybridization of the second agent, a labeling species 418u contacts the sample and reacts with reactive species 414u, depositing labeling species 418u (or a derivative thereof) at locations in the sample proximate to target analyte 410u. Following deposition of labeling species 418u, the second agent (e.g., including second oligonucleotide 412u and reactive species 414u) is removed from the sample by de-hybridization and washing.

To analyze target analyte 410u, light emitted from, reflected from, or transmitted from the sample is measured, and the contribution to the measured signal from labeling species 418u is determined to identify, and optionally quantify, target analyte 410u.

The sequence of steps described above to analyze target analyte 410u is then repeated to analyze target analytes 410v and 410w, with second agents that include different second oligonucleotides that specifically hybridize to first agents specifically bound to target analytes 410v and 410w. In the example shown in FIGS. 4D and 4E, two additional cycles of the sequence of steps described for target analyte 410u are performed to analyte target analytes 410v and 410w, respectively.

### Kits and Compositions

The agents, species, and moieties described herein can be included in a variety of kits featuring compositions that include the agents, species, and moieties. In general, a kit is a package of one or more reagents, each of which is in the form of a composition. Compositions featuring any of the different agents, species, and moieties described herein can be prepared and used for target analyte analysis as described herein. These compositions can be included in product kits, along with other features such as instructions for preparing compositions, and using the compositions for sample analysis. Product kits can be sealed or otherwise contained in a variety of different containers

### Measurement of Optical Signals Corresponding to Labeling Moieties

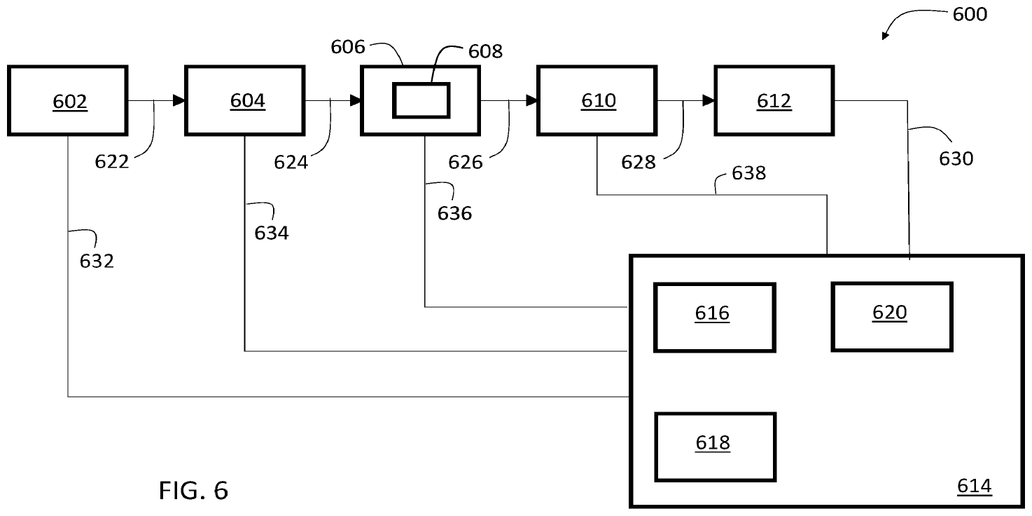

FIG. 6 is a schematic diagram showing a system 600 for acquiring multiple spectrally resolved images of a sample. System 600 can measure light emitted from, transmitted from, and/or reflected by a sample that includes one or more of the labeling moieties described herein. The measured light generally includes contributions from each of the labeling moieties present in the sample, and system 600 can analyze the multispectral image information encoded in the measured light, decomposing the image information to isolate contributions to the measured light from each of the labeling moieties in the sample. The decomposition yields, for each labeling moiety in the sample, a set of amplitude or intensity measurements as a function of position within the sample. The amplitude or intensity measurements can be used to quantify the amount of each labeling moiety, and therefore the amount of each target analyte, and each position in the sample.

A light source 602 provides light 622 to light conditioning optics 604. Light 622 can be incoherent light, such as light generated from a filament source for example, or light 622 can be coherent light, such as light generated by a laser. Light 622 can be either continuous-wave (CW) or time-gated (i.e., pulsed) light. Further, light 622 can be provided in a selected portion of the electromagnetic spectrum. For example, light 622 can have a central wavelength and/or a distribution of wavelengths that falls within the ultraviolet, visible, infrared, or other regions of the spectrum.

Light conditioning optics 604 can be configured to transform light 622 in a number of ways. For example, light conditioning optics 604 can spectrally filter light 622 to provide output light in a selected wavelength region of the spectrum. Alternatively, or in addition, light conditioning optics can adjust the spatial distribution of light 622 and the temporal properties of light 622. Incident light 624 is generated from light 622 by the action of the elements of light conditioning optics 604.

Incident light 624 is directed to be incident on sample 608 mounted on illumination stage 606. Stage 606 can provide means to secure sample 608, such as mounting clips or other fastening devices. Alternatively, stage 606 can include a movable track or belt on which a plurality of samples 608 are affixed. A driver mechanism can be configured to move the track in order to successively translate the plurality of samples, one at a time, through an illumination region on stage 606, whereon incident light 624 impinges. Stage 606 can further include translation axes and mechanisms for translating sample 608 relative to a fixed position of illumination stage 606. The translation mechanisms can be manually operated (e.g., threaded rods) or can be automatically movable via electrical actuation (e.g., motorized drivers, piezoelectric actuators).

In response to incident light 624, emitted light 626 emerges from sample 608. Emitted light 626 can be generated in a number of ways. For example, in some embodiments, emitted light 626 corresponds to a portion of incident light 624 transmitted through sample 608. In other embodiments, emitted light 626 corresponds to a portion of incident light 624 reflected from sample 608. In yet further embodiments, incident light 624 can be absorbed by sample 608, and emitted light 626 corresponds to fluorescence emission from sample 608 (e.g., from fluorescent components in sample 608) in response to incident light 624. In still further embodiments, sample 608 can be luminescent, and may produce emitted light 626 even in the absence of incident light 624. In some embodiments, emitted light 626 can include light produced via two or more of the foregoing mechanisms.

Light collecting optics 610 are positioned to received emitted light 626 from sample 608. Light collecting optics 610 can be configured to collimate emitted light 626 when light 626 is divergent, for example. Light collecting optics 610 can also be configured to spectrally filter emitted light 626. Filtering operations can be useful, for example, in order to isolate a portion of emitted light 626 arising via one of the mechanisms discussed above from light arising via other processes. For example, the methods described herein are used to determine accurate estimates of the fluorescence spectra of one or more labeling moieties in a sample. Light collecting optics 610 can be configured to filter out non-fluorescence components of emitted light 626 (e.g., components corresponding to transmitted and/or reflected incident light). Further, light collecting optics 610 can be configured to modify the spatial and/or temporal properties of emitted light 626 for particular purposes in embodiments. Light collecting optics 610 transform emitted light 626 into output light 628 which is incident on detector 612.

Detector 612 includes one or more elements such as CCD sensors configured to detect output light 628. In some embodiments, detector 612 can be configured to measure the spatial and/or temporal and/or spectral properties of light 628. Detector 612 generates an electrical signal that corresponds to output light 628, and is communicated via electrical communication line 630 to electronic control system 614.

Electronic control system 614 includes a processor 616, a display device 618, and a user interface 620. In addition to receiving signals corresponding to output light 628 detected by detector 612, control system 614 sends electrical signals to detector 612 to adjust various properties of detector 612. For example, if detector 212 includes a CCD sensor, control system 614 can send electrical signals to detector 612 to control the exposure time, active area, gain settings, and other properties of the CCD sensor.

Electronic control system 614 also communicates with light source 602, light conditioning optics 604, illumination stage 606, and light collecting optics 610 via electrical communication lines 632, 634, 636, and 638, respectively. Control system 614 provides electrical signals to each of these elements of system 600 to adjust various properties of the elements. For example, electrical signals provided to light source 602 can be used to adjust the intensity, wavelength, repetition rate, or other properties of light 622. Signals provided to light conditioning optics 604 and light collecting optics 610 can include signals for configuring properties of devices that adjust the spatial properties of light (e.g., spatial light modulators) and for configuring spectral filtering devices, for example. Signals provided to illumination stage 606 can provide for positioning of sample 608 relative to stage 606 and/or for moving samples into position for illumination on stage 606, for example.

Control system 614 includes a user interface 620 for displaying system properties and parameters, and for displaying captured images of sample 608. User interface 620 is provided in order to facilitate operator interaction with, and control over, system 600. Processor 616 includes a storage device for storing image data captured using detector 612, and also includes computer software that embodies instructions to processor 616 that cause processor 616 to carry out control functions, such as those discussed above for example. Further, the software instructions cause processor 616 to mathematically manipulate the images captured by detector 612 and to carry out the steps of decomposing images obtained by system 600 into contributions from particular labeling species in the sample.

In some embodiments, light conditioning optics 604 include an adjustable spectral filter element such as a filter wheel or a liquid crystal spectral filter. The filter element can be configured to provide for illumination of the sample using different light wavelength bands. Light source 602 can provide light 622 having a broad distribution of spectral wavelength components. A selected region of this broad wavelength distribution is allowed to pass as incident light 624 by the filter element in light conditioning optics 604, and directed to be incident on sample 608. Subsequently, the wavelength of the filter pass-band in light conditioning optics 604 is changed to provide incident light 624 having a different wavelength. Spectrally-resolved images can also be recorded by employing a light source 602 having multiple source elements generating light of different wavelengths, and alternately turning the different source elements on and off to provide incident light 624 having different wavelengths.

Light collecting optics 610 can include configurable spectral filter elements similar to those discussed above in connection with light conditioning optics 604. Therefore, spectral resolution can be provided on the excitation side of sample 608 (e.g., via light conditioning optics 604) and on the emission side of sample 608 (e.g., via light collecting optics 210).

The result of collecting multiple, spectrally resolved images of a sample is an “image stack” where each image in the stack is a two-dimensional image of the sample corresponding to a particular wavelength. Conceptually, the set of images can be visualized as forming a three-dimensional matrix, where two of the matrix dimensions are the spatial length and width of each of the images, and the third matrix dimension is the spectral index. For this reason, the set of spectrally resolved images can be referred to as a “spectral cube” of images. As used herein, a “pixel” in such a set of images (or image stack or spectral cube), refers to a common spatial location for each of the images. Accordingly, a pixel in a set of images includes a value associated with each image at the spatial location corresponding to the pixel.

To isolate contributions from each of multiple labeling species in a sample to the image information contained in a multispectral image stack, spectral unmixing methods can be used. Spectral unmixing is a technique that quantitatively separates contributions in an image that arise from spectrally different sources. For example, a sample may contain three different types of target analytes, each labeled with a labeling species. The three different labeling species may each have different absorption spectra. Typically, the individual absorption spectra of the labeling species are known before they are used, or they can be measured. Images of the sample under illumination will contain, in the most general case, spectral contributions from each of the three labeling species. A similar situation arises, for example, in samples containing multiple different fluorescent labeling species, each of which contributes to measured fluorescence emissions.

Spectral unmixing decomposes one or more images that include contributions from multiple spectral sources into a set of component images (the “unmixed images”) that correspond to contributions from each of the spectral entities within the sample. Thus, if the sample includes three different labeling species, each specific to a particular target analyte, then an image of the sample can be separated into three unmixed images, each unmixed image reflecting contributions principally from only one of the dyes.

The unmixing procedure essentially corresponds to decomposing an image into a set of spectral eigenstates. In many embodiments, the eigenstates are known beforehand, as discussed above. In other embodiments, the eigenstates can sometimes be determined using techniques such as principal component analysis. In either case, once the eigenstates have been identified, an image can be decomposed by calculating a set of values, usually as a coefficient matrix, that corresponds to the relative weighting of each of the eigenstates in the overall image. The contributions of each of the individual eigenstates can then be separated out to yield the unmixed image set.

As an example, a series of two dimensional images having x and y coordinates can be measured for a sample by illuminating the sample at a set of different excitation wavelengths λₖ. As described above, the two dimensional images can be combined to form a three-dimensional image cube I(x,y,k) where the first two indices of the image cube represent coordinate directions, and the third index is a spectral index corresponding to the wavelength of the illumination light. Assuming, for the sake of simplicity, that each of the images of the sample contains spectral contributions from two different spectral sources F(λₖ) and G(λₖ), then the values in the three-dimensional image cube I(x,y,k) may be given by

S(x,y,k)=a(x,y)·F(λₖ)+b(x,y)·G(λₖ) (1)

where λₖ is used to denote a given wavelength (or wavelength band). The functions a(x,y) and b(x,y) describe the spatial abundance of the spectral contributions from the two different spectral sources in the sample.

According to Equation (1), the net signal any position in the three-dimensional image cube (i.e., at any two-dimensional pixel coordinate, and at a particular illumination wavelength) is the sum of two contributions, weighted by the relative abundance of each. This can be expressed as

I(λₖ)=aF(λₖ)+bG(λₖ) (2)

The functions F and G can be termed the “spectral eigenstates” for the system because they correspond to the pure spectra for the spectral sources in the sample, which are combined in varying proportions to produce the measured spectral images of the sample. Thus, the sample spectrum is a weighted superposition corresponding to separate contributions from the two spectral sources.

If the spectra F(λₖ) and G(λₖ) are known (or can be deduced), then Equation (2) can be inverted to solve for a and b, provided that spectrum I includes at least two elements (i.e., provided that one has data for at least two wavelengths λₖ). Equation (2) can be rewritten in matrix form as I=EA, so that

A=E⁻¹I (3)

where A is a column vector with components a and b, and E is a matrix whose columns are the spectral eigenstates, namely [F G].

Using Equation (3), measured spectral images of a sample can be used to calculate contributions to the images arising purely from source F and purely from source G at particular pixel locations. The process can be repeated for each pixel location on a selected image (i.e., throughout the range of values x and y in I) to produce an image of the sample that includes contributions only from source F, and another image of the sample that includes contributions only from source G.

In the above discussion, the number of spectral sources is two (i.e., F and G). In general, however, unmixing techniques are not restricted to any particular number of sources. For example, a sample can generally contain m different spectral sources. If the number of wavelengths at which data is collected is n—that is, k=1 . . . n—then matrix E is an n×m matrix instead of an n×2 matrix, as in the above discussion. The unmixing algorithm can then be employed in the same manner as described above to isolate specific contributions at each pixel location in an image from each of the m spectral eigenstates.

One factor which can limit the ability of the algorithm to distinguish between contributions from different spectral eigenstates is the degree of spectral distinction between the eigenstates. The correlation between two spectra, such as two spectral eigenstates I₁ and I₂, can be described by a spectral angle θ where

$$θ = {cos}^{- 1} [ \frac{{I}_{1} \cdot {I}_{2}}{\vert {I}_{1} \vert \vert {I}_{2} \vert} ] \qquad ( 4 )$$

Sets of spectra for which θ is small for two members are not as easily separated into their components. Physically, the reason for this is easily understood: if two spectra are only marginally different, it is harder to determine the relative abundance of each.

A number of techniques can be used to measure or estimate the pure spectra of the spectral sources F and G (and other spectral sources, where the sample includes more than two). In general, any method that yields spectral eigenstates of sufficient accuracy can be used. Some samples can contain spectral sources such as dyes or other chemical moieties for which there are known spectra available in published reference materials. Alternatively, it may be possible to directly measure the spectra of source components using one or more measurement systems. In some samples, a particular region of the sample may be known to include only one particular spectral source, and the spectrum of that source can be extracted from measurements taken on only the identified region of the sample.

Various data analysis techniques can also be used for determining component spectra for spectral unmixing, such as principal component analysis (PCA), which identifies the most orthogonal spectral eigenvectors from an image cube and yields score images showing the weighting of each eigenvector throughout the image. This may be done in combination with other mathematical processing, and there are other known techniques for identifying low-dimensionality spectral vectors, such as projection pursuit, a technique described, for example, in L. Jimenez and D. Landgrebe, “Hyperspectral Data Analysis and Feature Reduction Via Projection Pursuit”, IEEE Transactions on Geoscience and Remote Sensing, Vol. 37, No. 6, pp. 2653-2667, November 1999, the entire contents of which are incorporated herein by reference. Other techniques include independent component analysis (ICA) and end-member detection algorithms, for example.

These techniques are typically not well-suited to the applications in the life sciences. For example, some techniques are optimized for spectral imaging data sets that contain spectra with dense spectral shapes and well-defined narrow peaks. In some techniques the spectral ranges are large compared to the individual spectral features and peaks that are used for analysis. The presence of peaks, or the ratio of peaks may be then used to classify “end-members” to be separated. Unfortunately, the components in biological samples typically do not have such well-defined, narrow peaks.

Some of these techniques generate images related to spectra that are present in a pure form somewhere within the original image cube. In many cases in the life sciences, signal spectra present in the image cube are mixtures of components. If the component of interest is not in a pure form somewhere in the original image cube, then it is unlikely that these techniques will generate an image that accurately represents the abundance of the component of interest.

There are some techniques, sometimes called “convex-hull” algorithms, that estimate what the true end-members are even if they do not exist in a pure form in the image, but the effectiveness is dependent on how close signal spectra in the image cube are to the end-members.

One technique that can be used to extract spectral eigenstates (or representations thereof) without a priori knowledge of all of the eigenstates involves considering the signal spectrum I(λₖ) for a given pixel, and subtracting from it the maximum amount of a first spectral source F(λₖ) while leaving the remaining signal that is positive definite in all spectral channels. That is, one defines a so-called “remainder spectrum” Uₐ(λₖ) for each pixel as

Uₐ(λₖ)=I(λₖ)−aF(λₖ) (5)

and then selects the largest value of the parameter a consistent with Uₐ(λₖ) having a non-negative value in every spectral channel. The resulting spectrum Uₐ(λₖ) is then used as the signal spectrum, expunged of contributions due to first spectral source F. One may also make the determination of parameter a based not on strict non-negative criterion listed above, but on some related criteria that incorporates a small negative distribution, to account for considerations such as shot noise or detector noise in a measurement system. Additional examples of optimization criteria for removing the maximal amount of spectral source F include using different error functions.

Alternatively, one may seek to extract a contribution to a measured spectrum that is due to second spectral source G. In analogy with Equation (5), the remainder spectrum can be calculated for each pixel as

U_{b}(λₖ)=I(λₖ)−bG(λₖ) (6)

where one selects the largest value of the parameter b consistent with U_{b}(λₖ) having a non-negative value in every spectral channel.

The remainder technique can be expanded to cases where the spectra for one or more additional components of the sample are known, and one wants to remove their contributions to the signal. In such cases, the remainder spectrum is written to subtract a contribution of each such component from the observed signal based on the additional spectra and consistent with a positive remainder in each spectral channel.

Additional aspects of spectral unmixing are described in U.S. Pat. Nos. 10,126,242 and 7,555,155, and in PCT Patent Publication No. WO2005/040769, the entire contents of each of which are incorporated herein by reference.

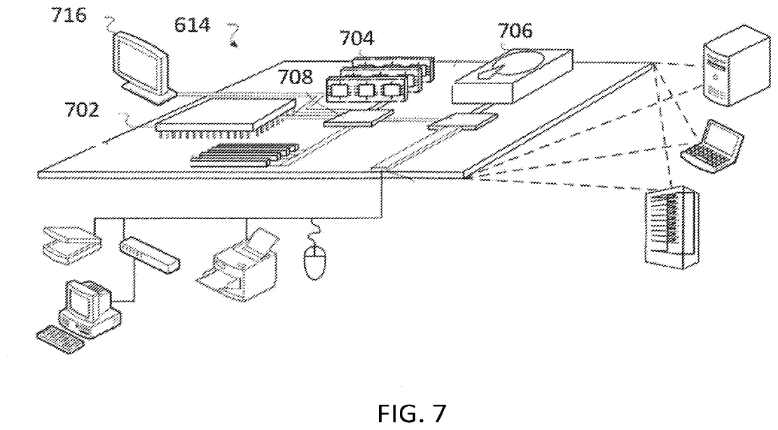

FIG. 7 shows an example of an electronic control system 614, which may be used with the systems and methods disclosed herein. Electronic control system can include one or more processors 702 (e.g., corresponding to processor 616 in FIG. 6), memory 704, a storage device 706 and interfaces 708 for interconnection. The processor 702 can process instructions for execution within the electronic control system 614, including instructions stored in the memory 704 or on the storage device 706. For example, the instructions can instruct the processor 702 to perform any of the analysis and control steps disclosed herein.

The memory 704 can store executable instructions for processor 702, information about parameters of the system such as excitation and detection wavelengths, and measured spectral image information. The storage device 706 can be a computer-readable medium, such as a floppy disk device, a hard disk device, an optical disk device, or a tape device, a flash memory or other similar solid state memory device, or an array of devices, including devices in a storage area network or other configurations. The storage device 706 can store instructions that can be executed by processor 702 described above, and any of the other information that can be stored by memory 704.

In some embodiments, electronic control system 614 can include a graphics processing unit to display graphical information (e.g., using a GUI or text interface) on an external input/output device, such as display 716. The graphical information can be displayed by a display device (e.g., a CRT (cathode ray tube) or LCD (liquid crystal display) monitor) for displaying any of the information, such as measured and calculated spectra and images, disclosed herein. A user can use input devices (e.g., keyboard, pointing device, touch screen, speech recognition device) to provide input to the electronic control system 614.

The methods disclosed herein can be implemented by electronic control system 614 (and processors 702 and 616) by executing instructions in one or more computer programs that are executable and/or interpretable on the electronic control system 614. These computer programs (also known as programs, software, software applications or code) include machine instructions for a programmable processor, and can be implemented in a high-level procedural and/or object-oriented programming language, and/or in assembly/machine language. For example, computer programs can contain the instructions that can be stored in memory 704, in storage unit 706, and/or on a tangible, computer-readable medium, and executed by processor 702 (processor 616) as described above. As used herein, the term “computer-readable medium” refers to any computer program product, apparatus and/or device (e.g., magnetic discs, optical disks, memory, Programmable Logic Devices (PLDs), ASICs, and electronic circuitry) used to provide machine instructions and/or data to a programmable processor, including a machine-readable medium that receives machine instructions.

Generally, electronic control system 614 can be implemented in a computing system to implement the operations described above. For example, the computing system can include a back end component (e.g., as a data server), or a middleware component (e.g., an application server), or a front end component (e.g., a client computer having a graphical user-interface), or any combination thereof.

### Reagents and Conditions

In general, the various steps described herein can be implemented under a wide variety of conditions and with different reagents. Accordingly, the reagents and conditions described in this section should be understood to represent only examples of suitable reagents and conditions.

Typically, the first agent can be stored following preparation in a buffer solution that can include one or more of PBS, PBS-T, TBS, TBS-T, water, saline solution, and Kreb's buffer. The buffer solution can optionally include one or more blocking materials. Examples of suitable blocking materials include, but are not limited to, BSA, casein, sheared salmon-sperm DNA, oligonucleotides, rat IgG antibodies, and mouse IgG antibodies.

The second agent can also be stored following preparation in a buffer solution. The buffer can include one or more of PBS, PBS-T, TBS, TBS-T, water, saline solution, and Kreb's buffer. The buffer solution can be the same as, or different from, the buffer solution used to store the first agent.

To promote hybridization between the first and second agents and/or between the first agent and a reporting agent, the first and second agents (or first agent and reporting agent) can be immersed in a hybridization buffer. Suitable hybridization buffers can include DNA components, protein components, detergents, and/or chaotropic reagents at concentrations of between 5% and 20%.

To promote de-hybridization between the first and second agents and/or between the first agent and a reporting agent, the first and second agents (or first agent and reporting agent) can be immersed in a de-hybridization buffer. Suitable hybridization buffers can include chaotropic reagents such as DMSO and/or formamide, at concentrations of between 60% and 90%.

To promote binding of the first agent to a target analyte in a sample, the first agent can be layered onto the sample in solution, e.g., by pipetting, and incubated with the sample. Following incubation, unbound first agent can be washed from the sample using, for example, a buffer solution that includes one or more of PBS, PBS-T, TBS, TBS-T, water, saline solution, and Kreb's buffer.

The incubation time for any of the hybridization, reaction, binding, and de-hybridization steps described herein can be 10 minutes or more (e.g., 20 minutes or more, 30 minutes or more, 40 minutes or more, 60 minutes or more, 1 hour or more, 2 hours or more, 3 hours or more, 4 hours or more, 5 hours or more, 6 hours or more, 8 hours or more, 10 hours or more, 16 hours or more, 20 hours or more, 24 hours or more, 48 hours or more, 7 days or more, 30 days or more).

### Examples

To demonstrate the efficacy of the methods described herein for analyzing multiple target species in a biological sample, several investigations were conducted. First, a FFPE sample of human tonsil tissue was obtained. In a first labeling and imaging cycle, the tissue sample was labeled with a first agent that included an antibody binding agent specifically targeting the biomarker PD-1. A second agent was then incubated with the sample, and hybridized to the first agent. The second agent included an oligonucleotide-conjugated HRP moiety. A labeling agent that included a labeling moiety (OPAL® dye HX0046, available from Akoya Biosciences, Inc., Menlo Park, Calif.) was deposited in the tissue sample via HRP-mediated TSA. Following removal of the second agent, the sample was imaged to reveal the presence of PD-1.

Two more analytical cycles were performed. In the second cycle, the sample was labeled with a first agent that included an antibody binding agent specifically targeting the biomarker PDL1, and a labeling moiety (OPAL® dye HX043, available from Akoya Biosciences, Inc.) was deposited via HRP-mediated TSA to label PDL1. In the third cycle, the sample was labeled with a first agent that included an antibody binding agent specifically targeting the biomarker FOXP3, and a labeling moiety (OPAL® dye HX031, available from Akoya Biosciences, Inc.) was deposited via HRP-mediated TSA to label FOXP3.

FIGS. 8A-8C are images showing the distribution and relative concentrations of biomarkers PD-1, PDL1, and FOXP3, respectively, in the tissue sample. FIG. 8D is an overlay image showing the distributions of all three markers within the sample. As is evident from the images, each of the different biomarkers can be independently identified and quantified in the sample, with little or no cross-channel interference when the applied labeling moieties are suitably chosen.

To investigate the effect of amplification via HRP-mediated TSA, a human FFPE section of tonsil tissue was obtained, and more than fifteen different first agents (each with a binding agent that targeted a different biomarker) were bound to the sample. Corresponding reporting agents were then hybridized to the first agents, and images of the sample with subsets of the different reporting agents were obtained. The reporting agents were then removed from the sample via de-hybridization, as described above, and labeling moieties targeting three different markers—PD-1, PDL-1, and FOXP3—were deposited in the sample in three separate HRP-mediated TSA labeling cycles. An image of the sample with signals corresponding to markers PD-1, PDL-1, and FOXP3 amplified was then obtained.

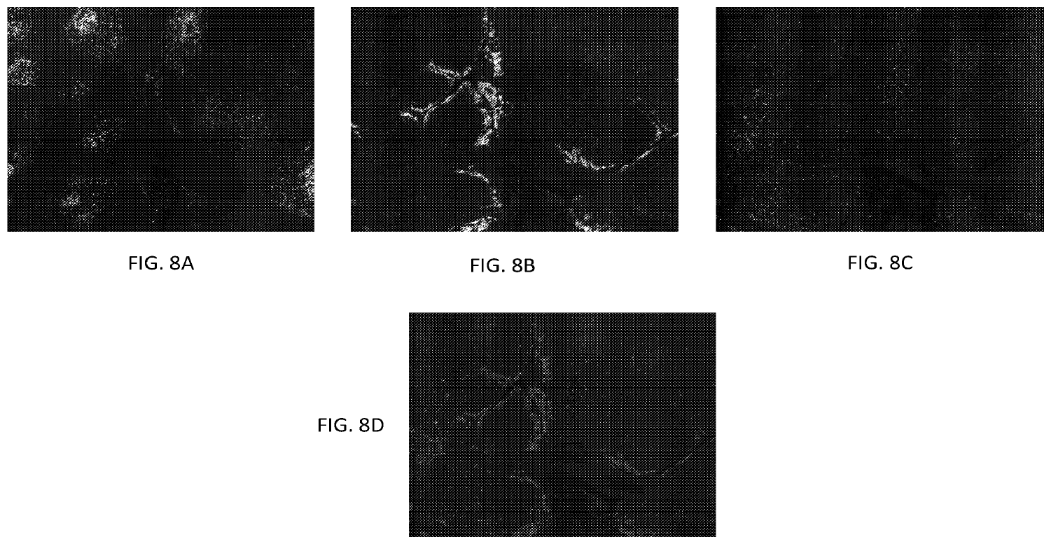

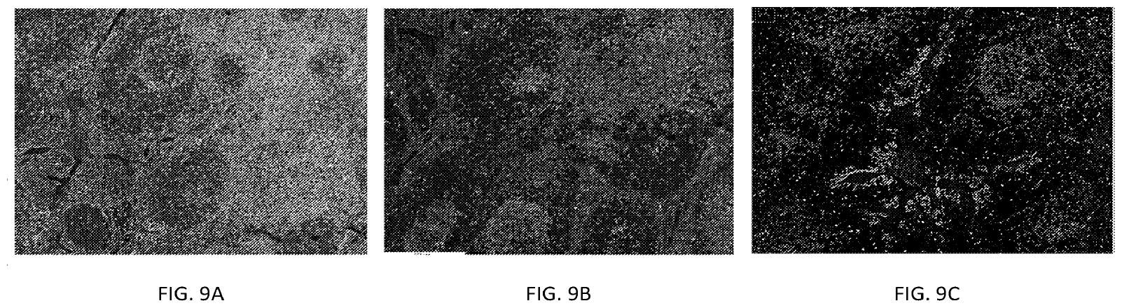

FIG. 9A is an image showing the distributions and relative concentrations of markers CD8, CD31, CD20, CD45RO, CD4, Pancytokeratin, and CD34 in the tissue section, and FIG. 9B is an image showing the distributions and relative concentrations of CD11c, Ki67, PDL-1, E-cadherin, CD3, and FOXP3 in the tissue section. Amplified signals due to PD-1, PDL-1, and FOXP3 are shown in the image of FIG. 9C. Signals corresponding to PDL-1 and FOXP3 in FIG. 9C are of significantly larger intensity than corresponding signals for these markers in FIG. 9B. Nucleotide sequences corresponding to the first agent (first oligonucleotide), the reporting agent, and the second agent (second oligonucleotide) are shown in Table 1 below.

TABLE 1

| | First | Reporting | Second |
|---|---|---|---|
| Binding | Agent | Agent | Agent |
| Agent | Oligo- | Oligo- | Oligo- |
| Target | nucleotide | nucleotide | nucleotide |
| Analyte | Sequence | Sequence | Sequence |
| CD8- | TTCTCACTAC | AGTACTAATA | |
| BX030 | TATTAGTACT | GTAGTGA | |
| CD31- | GATTGGTCCA | TACGTTAGTG | |
| BX001 | CTAACGTA | GACCA | |
| CD20- | TCCAACCAAA | CGGTCAGTTT | |
| BX007 | CTGACCG | GGTT | |
| CD45RO- | TCGCAAGGAA | TCGGTAGTTC | |
| BX017 | CTACCGA | CTTG | |
| CD4- | AGGTGGATGT | ATCGTAACAC | |
| BX003 | GTTACGAT | ATCCA | |
| PanCK- | CGCGGTTTCC | TGTCTGAGGA | |
| BX019 | TCAGACA | AACC | |
| CD34- | GTACACATGA | TGATTCGCTC | |
| BX020 | GCGAATCA | ATGTG | |
| CD11c- | CACTCTCCCA | CCGACTAATG | |
| BX024 | TTAGTCGG | GGAGA | |
| Ki67- | GCCGACGACG | GCCTTCGTCG | |
| BX047 | AAGGC | TC | |
| PDL1- | GCCCGCTAAG | | CTTAGAATAT |
| BX026 | ATATTCTAAG | | CTTAGCG |
| E- | GACAACGCGA | ATCCGTCGCG | |
| cadherin- | CGGAT | TT | |
| BX014 | | | |
| CD3- | GTTGATAAAT | ACTCTGTAAA | |
| BX045 | ATTTTACAGA | ATATTTATC | |
| | GT | | |
| FOXP3- | GACGCTTATT | | GAAGTCCATA |
| BX015 | ATGGACTTC | | ATAAGC |
| PD-1- | ACAAAGCGGT | | ACCGTAAGAC |
| BX006 | CTTACGGT | | CGCTT |

To demonstrate removal of the second agent from a sample, a FFPE section of tonsil tissue was obtained and subjected to three cycles of HRP-mediated TSA to deposit labeling moieties. In the first cycle, OPAL® dye 570 (Akoya Biosciences, Inc.) was deposited in the sample via HRP-mediated TSA. The second agent was then removed from the sample, and a second cycle was conducted by introducing OPAL® dye 690 (Akoya Biosciences, Inc.) with no second agent hybridized, and therefore no HRP reactive agent present in the sample. In the third cycle, OPAL® dye 690 was deposited via HRP-mediated TSA. Sample images were obtained following the second and third cycles.

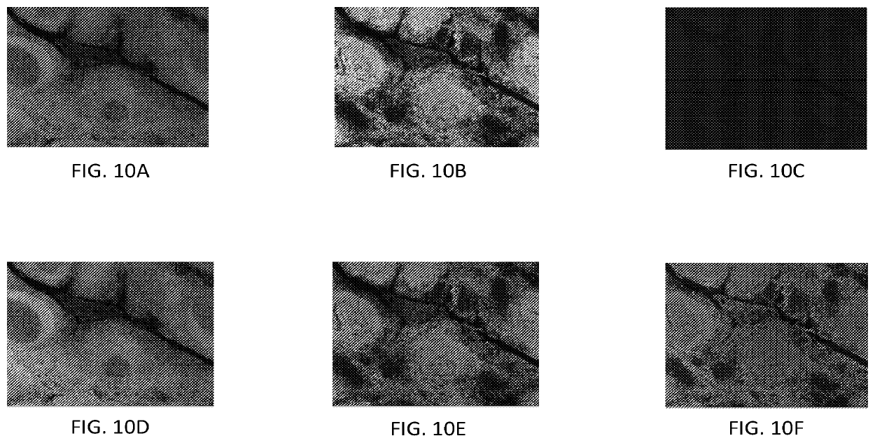

FIGS. 10A and 10D show nuclear staining images after cycles 2 and 3, FIGS. 10B and 10E show OPAL® dye 570 images after cycles 2 and 3, and FIGS. 10C and 10F show OPAL® dye 690 images after cycles 2 and 3. As is evident from FIGS. 10C and 10F, complete removal of the second agent was achieved between cycles 1 and 2.

For the foregoing examples, the labeling protocol for OPAL® dyes 570 and 670 was as follows:
(a) Wash the tissue 3 times with 20% DMSO.
(b) Incubate the tissue for 10 min with 200 μl of hybridization buffer. After 10 min, wash the tissue with 20% DMSO (3×) followed by 3× wash with 1× CODEX® assay buffer.
(c) Add 200 μl of OPAL® dyes (O570 or O670) diluted in 1× plus amplification dilution reagent in the ratio of 1:200 and 1:400 respectively. Incubate for 20 min.
(d) After 20 min, wash the tissue with 1× CODEX® assay buffer (3×). Wash with DI water (3×) and image at 20× objective.

The labeling protocol for OPAL® dye 780 was as follows:
(a) Wash the tissue 3 times with 20% DMSO. Incubate the tissue for 10 min with 200 ul of Hybridization buffer. After 10 min, wash the tissue with 20% DMSO (3×) followed by 3× wash with 1× CODEX® assay buffer.
(b) Add 200 ul of TSA dig diluted in the ratio of 1:50 with 1× plus amplification dilution reagent. Incubate for 15 min.
(c) After 15 min, wash the tissue with 1× CODEX® assay buffer.
(d) Add 200 ul of OPAL 780 diluted in 1× plus amplification dilution reagent in the ratio of 1:50. Incubate for 1 hr. Wash with 1× CODEX® assay buffer (3×). Wash with DI water (3×) and image at 20× objective.

### Other Embodiments

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

In addition to the embodiments expressly disclosed herein, it will be understood that various modifications to the embodiments described may be made without departing from the spirit and scope of the disclosure. Accordingly, other embodiments are within the scope of the following claims.

What is claimed is:

1. A method, comprising:
(i) contacting a biological sample comprising a first target analyte with a first agent, wherein the first agent comprises a first binding species that specifically binds to the first target analyte, and a first oligonucleotide conjugated to the binding species;
(ii) contacting the biological sample with a second agent, wherein the second agent comprises a first reactive species and a second oligonucleotide conjugated to the first reactive species, to hybridize at least a portion of the second oligonucleotide to at least a portion of the first oligonucleotide;
(iii) contacting the biological sample with a first labeling species, wherein the first labeling species reacts with the first reactive species to deposit the first labeling species or a derivative thereof in the biological sample;
(iv) removing the second agent from the biological sample following deposition of the first labeling species or the derivative thereof;
(v) contacting the biological sample with a third agent, wherein the third agent comprises a second binding species that specifically binds to a second target analyte in the biological sample, and a third oligonucleotide conjugated to the second binding species;
(vi) contacting the biological sample with a fourth agent, wherein the fourth agent comprises a second reactive species and a fourth oligonucleotide conjugated to the second reactive species, to hybridize at least a portion of the fourth oligonucleotide to at least a portion of the third oligonucleotide; and
(vii) contacting the biological sample with a second labeling species, wherein the second labeling species reacts with the second reactive species to deposit the second labeling species or a derivative thereof in the biological sample.

2. The method of claim 1, wherein the first reactive species comprises a catalytic agent.

3. The method of claim 1, wherein the first reactive species comprises an enzyme.

4. The method of claim 3, wherein the enzyme comprises horseradish peroxidase.

5. The method of claim 1, wherein the first labeling species comprises a dye.

6. The method of claim 4, wherein the first labeling species comprises a conjugate of an inactive tyramide or a derivative thereof and a dye.

7. The method of claim 6, wherein contacting the biological sample with the first labeling species comprises converting the first labeling species to a conjugate of an active tyramide or a derivative thereof and the dye, wherein the active tyramide or a derivative thereof binds to the biological sample in proximity to the second agent.

8. The method of claim 1, wherein the first binding species comprises an antibody or an antibody fragment.

9. The method of claim 1, wherein the first oligonucleotide comprises at least 10 nucleotides.

10. The method of claim 1, wherein the second oligonucleotide comprises at least 10 nucleotides.

11. The method of claim 1, wherein nucleotide sequences of the first and second oligonucleotides are at least 70% complementary.

12. The method of claim 1, wherein the second oligonucleotide comprises a larger number of nucleotides than the first oligonucleotide.

13. The method of claim 1, wherein the second oligonucleotide comprises multiple contiguous, non-consecutive nucleotide sequences that are complementary to different portions of a sequence of the first oligonucleotide.

14. The method of claim 1, wherein the first and second reactive species are the same.

15. The method of claim 1, wherein the first and second reactive species each comprise an enzyme.

16. The method of claim 1, wherein the first and second reactive species each comprise horseradish peroxidase.

17. The method of claim 1, wherein the first and third oligonucleotides are different.

18. The method of claim 1, wherein the second and fourth oligonucleotides are different.

19. The method of claim 1, wherein the first labeling species comprises a first dye, and wherein the second labeling species comprises a second dye different from the first dye.

20. The method of claim 1, wherein the first binding species comprises a first antibody or a first antibody fragment, and wherein the second binding species comprises a second antibody or a second antibody fragment, and wherein the first and second binding species selectively bind to different first and second target analytes in the biological sample.

21. The method of claim 1, wherein the first oligonucleotide comprises a nucleotide sequence of RNA bases.

22. The method of claim 1, wherein the first oligonucleotide comprises a nucleotide sequence of DNA bases.

23. The method of claim 1, wherein the first oligonucleotide comprises at least one synthetic nucleotide.

24. The method of claim 1, wherein the first oligonucleotide is fully single-stranded.

25. The method of claim 1, wherein the first oligonucleotide is partially double-stranded.

26. The method of claim 5, wherein the dye comprises a chromogenic species or a fluorescent species.

27. A reagent kit, comprising:
a first agent, wherein the first agent comprises a first binding species that specifically binds to a first target analyte of a biological sample, and a first oligonucleotide conjugated to the first binding species;
a second agent, wherein the second agent comprises a second binding species that specifically binds to a second target analyte of the biological sample, and a second oligonucleotide conjugated to the second binding species;
a third agent, wherein the third agent comprises a reactive species and a third oligonucleotide conjugated to the reactive species;
a fourth agent, wherein the fourth agent comprises the reactive species and a fourth oligonucleotide conjugated to the reactive species;
a first labeling species; and
a second labeling species,
wherein the first and second labeling species each react with the reactive species to deposit the first and second labeling species or a derivative thereof, respectively, in the biological sample.
